(19) United States
(12) Patent Application Publication
(10) Pub. No.: US 2017/0242189 A1
(43) Pub. Date: Aug. 24, 2017

(54) LOW-RESISTANCE CLADDING MATERIAL AND ELECTRO-OPTIC POLYMER OPTICAL WAVEGUIDE

(71) Applicants: KYUSHU UNIVERSITY, Fukuoka-shi, Fukuoka (JP); NISSAN CHEMICAL INDUSTRIES, LTD., Tokyo (JP)

(72) Inventors: Shiyoshi YOKOYAMA, Fukuoka-shi (JP); Kazuhiro YAMAMOTO, Fukuoka-shi (JP); Hiromu SATO, Fukuoka-shi (JP); Daisuke MAEDA, Funabashi-shi (JP); Masaaki OZAWA, Funabashi-shi (JP); Kei YASUI, Funabashi-shi (JP)

(73) Assignees: KYUSHU UNIVERSITY, Fukuoka-shi, Fukuoka (JP); NISSAN CHEMICAL INDUSTRIES, LTD., Tokyo (JP)

(21) Appl. No.: 15/508,066

(22) PCT Filed: Sep. 2, 2015
(86) PCT No.: PCT/JP2015/074969

§ 371 Date: Apr. 19, 2017

(30) Foreign Application Priority Data
Sep. 2, 2014 (JP) 2014-178464
Aug. 7, 2015 (JP) 2015-157375

(51) Int. Cl. G02B 6/122; C08K 3/04; G02B 6/138; C07F 7/18; G02F 1/01; G02B 6/136; C08K 5/092; C07D 409/06

(52) U.S. Cl. CPC G02B 6/122; C08K 5/092; C08K 3/041; C07D 409/06; C07F 7/1844; G02F 1/011; G02B 6/136; G02B 6/138; G02B 2006/12142

(57) ABSTRACT

An optical waveguide which has sufficient orientation characteristics and its manufacturing processes are simple to be suitable for the manufacture of electro-optic elements and that can be reduced the power consumption by its large electro-optic characteristics and further can be thinned and stacked, and the material thereof. This material is characterized in a polymer compound that includes an oxazoline structure in a side chain, and an acid generator or a polyvalent carboxylic acid.

### TECHNICAL FIELD

The present invention relates to an optical waveguide containing an organic nonlinear optical compound for use in optical information processing, optical communication, and the like such as optical switches and optical modulation.

### BACKGROUND ART

Devices such as optical modulators and optical switches use a nonlinear optical effect, especially an electro-optic effect, in which a refractive index changes by an electric field. Although inorganic materials such as lithium niobate and potassium dihydrogenphosphate have been conventionally widely used as the nonlinear optical material exhibiting this effect, organic nonlinear optical materials have been receiving attention in order to satisfy demands for more advanced nonlinear optical performance, a reduction in manufacturing costs, and the like, and studies for their practical use have been becoming active.

In particular, optical waveguide modulators to which electro-optic polymer materials having electro-optic characteristics extremely higher than conventional inorganic materials are applied have been developed, which is boosting expectations for the achievement of ultrahigh-speed modulation devices and low power consumption device technology. Optical modulators manufactured using these polymer materials are superior to optical waveguide modulators using conventional inorganic crystals in low voltage operation caused by high electro-optic characteristics in the polymer materials and favorable high-frequency control caused by low dielectric constant characteristics.

To achieve optical waveguide modulators with low voltage operation, the electro-optic constant (r₃₃) of the electro-optic polymer materials is required to be increased, and materials exceeding r₃₃=100 pm/V have been so far developed.

An optical waveguide required when the nonlinear optical material is used for light propagation type devices is formed as a stacked structure having a polymer core containing the nonlinear optical compound and a cladding that is formed on and under or around the core and has a refractive index lower than that of the core. In this stacked structure, when electric field orientation is performed in the optical waveguide, in a stationary state, according to Ohm's law, voltage is applied in a divided manner in proportion to the electric resistivity of the individual layers. Consequently, to effectively apply voltage to the core, the electric resistivity of the core may be increased compared with that of the other layer (the cladding). However, when a π-electron conjugated dye having high hyperpolarizability is introduced into the polymer in a high concentration in order to obtain a high nonlinear optical effect, the electric resistivity of the core tends to decrease. Therefore, when a polymer material that can exhibit a large nonlinear optical effect is used for the core to manufacture an optical waveguide element, another cladding member having comparable resistivity or lower is required to be selected in order to perform high electric field orientation in the optical waveguide. However, most general-purpose optical polymers have a resistivity of 10⁹ Ωm or higher (Non-Patent Document 1), which is higher than the resistivity of nonlinear optical materials, which is 10⁷ to 10⁸ Ωm. Thus, in the optical waveguide structure having the nonlinear optical material in the core, voltage applied via upper and lower electrodes is concentrated on the cladding having higher resistivity, and efficient voltage application to the core cannot be achieved, making it difficult to increase the electric field orientation of the nonlinear optical compound of the polymer core. Consequently, high voltage of several hundred volts or higher has been required to be applied to optical waveguides in electric field orientation treatment.

To solve the problem, a method is reported that reduces the resistance value of the cladding and improves poling efficiency by adding a polymer compound having an alkylammonium group to the cladding material (Patent Document 1). Similarly, a method is also reported that reduces the resistance value of the cladding compared with the resistance value of the core by adding a nonlinear optical compound, which has been contained only in the core, also to the cladding (Patent Document 2).

### PRIOR ART DOCUMENTS

### Patent Documents

[Patent Document 1] Japanese Patent No. 3477863 (JP 3477863 B) specification

[Patent Document 2] International Publication WO 2013/024840

### PAMPHLET

### Non-Patent Documents

[Non-Patent Document 1] Appl. Phys. Lett. 90, 191103 (2007)

### SUMMARY OF THE INVENTION

### Problems to be Solved by the Invention

In the proposed methods described above, sufficient orientation characteristics have not yet been obtained, and voltage of several hundred volts or higher is required to be applied to optical waveguides in the electric field orientation treatment. Given these circumstances, desired are the development of a polymer cladding material for which manufacturing processes are simple to be suitable for the manufacture of electro-optic elements and that can obtain large electro-optic characteristics contributing to a reduction in the power consumption of the elements and can be thinned and stacked and an optical waveguide using the same.

### Means for Solving the Problem

In order to achieve the object, the inventors of the present invention have conducted intensive studies to find out that a cladding material containing a polymer compound containing an oxazoline structure in a side chain and an acid generator or a polycarboxylic acid can reduce the resistance value of the cladding compared with the resistance value of the core and that an optical waveguide modulator that is low in applied voltage for electric field orientation and is low in optical modulation operating voltage is achieved and have completed the present invention.

Specifically, according to a first aspect, the present invention relates to a cladding material of an optical waveguide comprising a polymer compound containing an oxazoline structure in a side chain and an acid generator or a polycarboxylic acid.

According to a second aspect, the present invention relates to the cladding material of an optical waveguide according to the first aspect, comprising the polymer compound containing an oxazoline structure in a side chain and the acid generator. According to a third aspect, the present invention relates to the cladding material of an optical waveguide according to the first aspect, comprising the polymer compound containing an oxazoline structure in a side chain and the polycarboxylic acid.

According to a fourth aspect, the present invention relates to the cladding material of an optical waveguide according to the first aspect, comprising the polymer compound containing an oxazoline structure in a side chain, a carbon nanotube, and the acid generator or the polycarboxylic acid.

According to a fifth aspect, the present invention relates to the cladding material of an optical waveguide according to the third aspect, comprising the polymer compound containing an oxazoline structure in a side chain, a carbon nanotube, and the polycarboxylic acid.

According to a sixth aspect, the present invention relates to the cladding material of an optical waveguide according to any one of the first aspect to the fifth aspect, in which the polymer compound is obtained by radically polymerizing at least two kinds of monomers of an oxazoline monomer having a polymerizable carbon-carbon double bond-containing group at the 2-position of an oxazoline ring and a hydrophilic functional group-containing (meth)acrylic monomer.

According to a seventh aspect, the present invention relates to an optical waveguide comprising a core and a cladding that surrounds an entire outer periphery of the core and has a refractive index lower than that of the core, the cladding being formed of the cladding material as described in any one of the first aspect to the sixth aspect.

According to an eighth aspect, the present invention relates to the optical waveguide according to the seventh aspect, in which the core contains an organic nonlinear optical compound having a tricyano-bonded furan ring of Formula [2] or a derivative thereof:

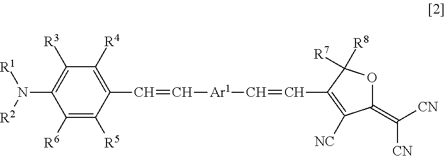

(where R^{′} and R² are each independently a hydrogen atom, a C₁₋₁₀ alkyl group optionally having a substituent, or a C₆-₁₀ aryl group optionally having a substituent; R³ to R⁶ are each independently a hydrogen atom, a C₁₋₁₀ alkyl group, a hydroxy group, a C₁₋₁₀ alkoxy group, a C₂₋₁₁ alkylcarbonyloxy group, a C₄₋₁₀ aryloxy group, a C₅₋₁₁ arylcarbonyloxy group, a silyloxy group having a C₁₋₆ alkyl group and/or phenyl group, or a halogen atom; R⁷ and R⁸ are each independently a hydrogen atom, a C₁₋₅ alkyl group, a C₁₋₅ haloalkyl group, or a C₆₋₁₀ aryl group; and Ar¹ is a divalent aromatic group of Formula [3] below or Formula [4] below):

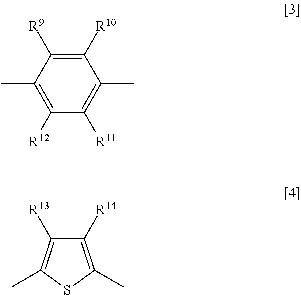

(where R⁹ to R¹⁴ are each independently a hydrogen atom, a C₁₋₁₀ alkyl group optionally having a substituent, or a C₆₋₁₀ aryl group optionally having a substituent).

According to a ninth aspect, the present invention relates to a method for manufacturing the optical waveguide as described in the eighth aspect including a core and a cladding that surrounds an entire outer periphery of the core and has a refractive index lower than that of the core, the method comprising:

forming a lower cladding using the cladding material as described in any one of the first aspect to the sixth aspect;

forming a core containing the nonlinear optical compound having a tricyano-bonded furan ring of Formula [2] or the derivative thereof as described in the eight aspect on the lower cladding;

forming an upper cladding using the cladding material as described in any one of the first aspect to the sixth aspect on the core; and

performing polarization orientation treatment on the nonlinear optical compound or the derivative thereof contained in the core before and/or after the forming the upper cladding.

According to a tenth aspect, the present invention relates to a method for manufacturing the ridge type optical waveguide as described in the eighth aspect including a core and a cladding that surrounds an entire outer periphery of the core and has a refractive index lower than that of the core, the method comprising:

forming a lower cladding using the cladding material as described in any one of the first aspect to the sixth aspect;

forming a resist layer having photosensitivity to ultraviolet rays or an electron beam on the lower cladding, irradiating a surface of the resist layer with ultraviolet rays via a photomask or directly irradiating a surface of the resist layer with an electron beam, performing development to form a mask pattern of the core, transferring a core pattern to the lower cladding with the mask pattern serving as a mask, and removing the resist layer;

forming a core containing the nonlinear optical compound having a tricyano-bonded furan ring of Formula [2] or the derivative thereof as described in the eighth aspect on the lower cladding;

forming an upper cladding using the cladding material as described in any one of the first aspect to the sixth aspect on the core; and

performing polarization orientation treatment on the nonlinear optical compound or the derivative thereof contained in the core before and/or after the forming the upper cladding.

According to an eleventh aspect, the present invention relates to the method for manufacturing according to the ninth aspect or the tenth aspect, in which the polarization orientation treatment is electric field application treatment by electrodes.

### Effects of the Invention

The cladding material of the present invention, showing a low resistivity, is used as a cladding of an optical waveguide and can thereby form an optical waveguide that can perform simple, efficient electric field application to a core having high nonlinear optical characteristics.

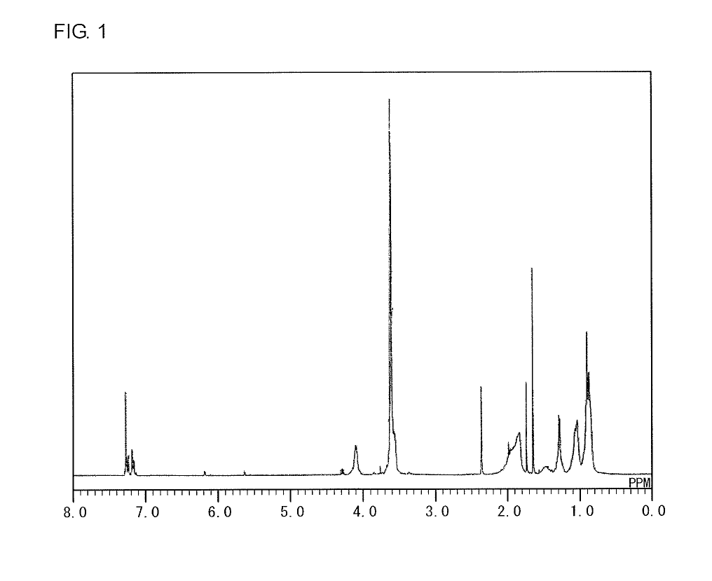

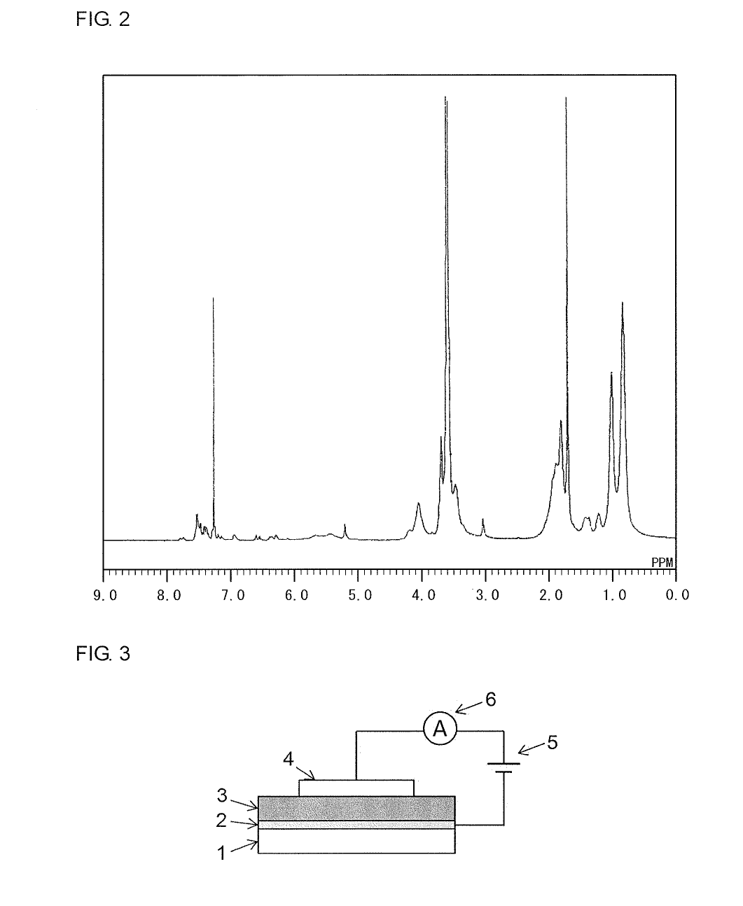

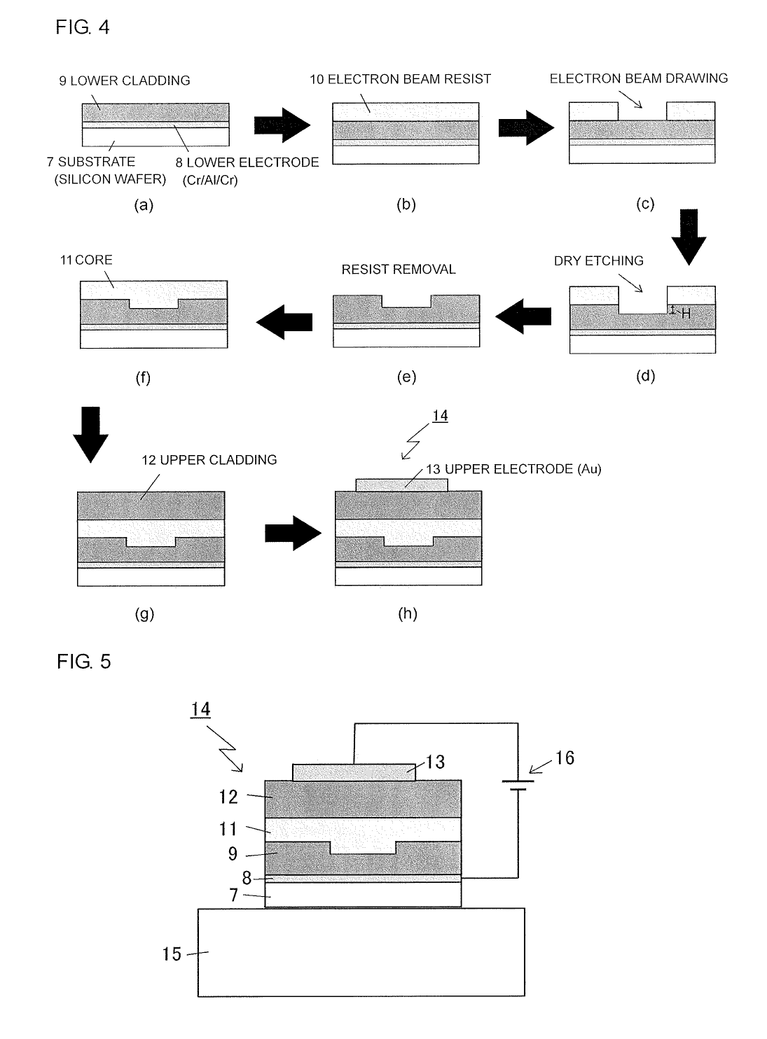

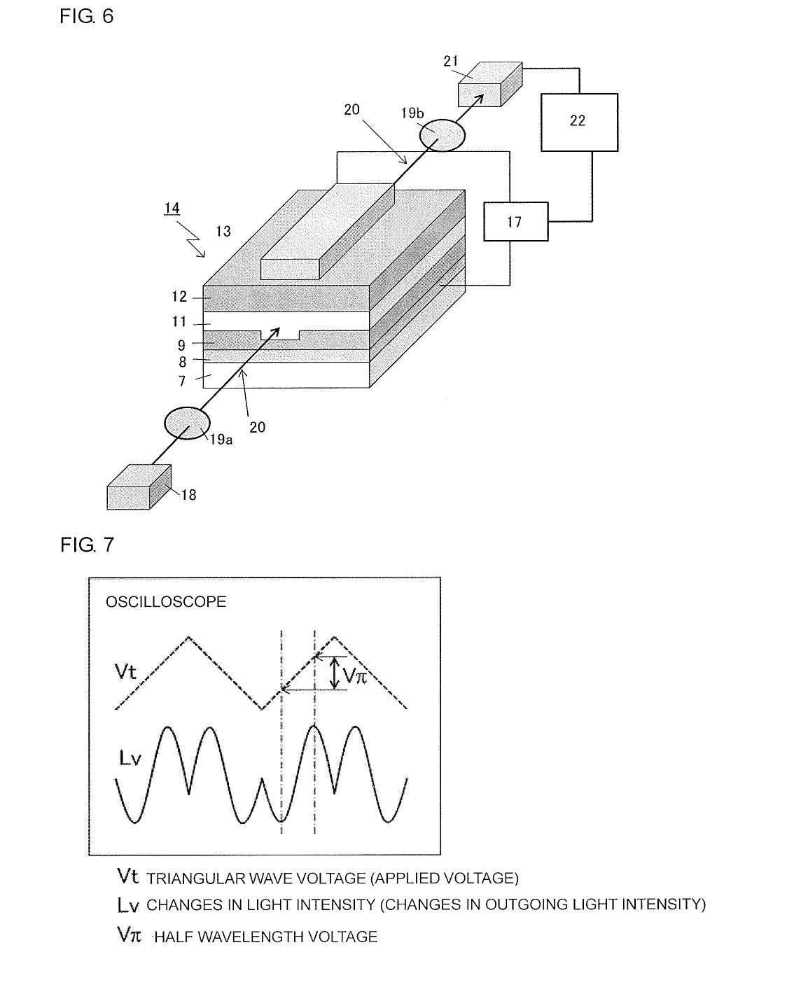

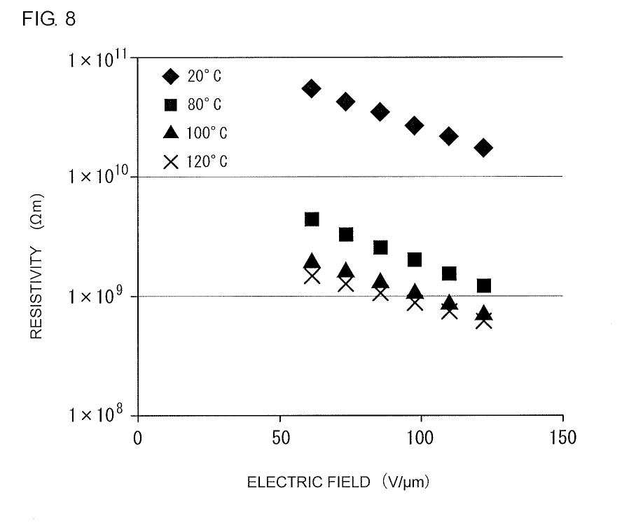

### BRIEF DESCRIPTION OF THE DRAWINGS

[FIG. 1] FIG. 1 is a diagram of a ¹HNMR spectrum of PcM manufactured in Manufacture Example 1-1.

[FIG. 2] FIG. 2 is a diagram of a ¹HNMR spectrum of PMC110-10 manufactured in Manufacture Example 1-2.

[FIG. 3] FIG. 3 is a diagram of a conceptual diagram of an apparatus used in resistivity measurement in Example 2.

[FIG. 4] FIG. 4 is a diagram of a process diagram of a process of manufacturing a ridge type optical waveguide manufactured in Example 3.

[FIG 5] FIG. 5 is a diagram of a conceptual diagram of an apparatus used for polarization orientation treatment on the ridge type optical waveguide manufactured in Example 3.

[FIG. 6] FIG. 6 is a diagram of a conceptual diagram of an apparatus used for characteristic analysis on the ridge type optical waveguide manufactured in Example 3.

[FIG. 7] FIG. 7 is a diagram of a relation among a triangular wave voltage (an applied voltage), changes in light intensity (changes in outgoing light intensity), and a half wavelength voltage (Vπ).

[FIG. 8] FIG. 8 is a diagram of the results of resistivity measurement in Example 5.

### MODES FOR CARRYING OUT THE INVENTION

A subject of the present invention is a cladding material of an optical waveguide comprising a polymer compound containing an oxazoline structure in a side chain and an acid generator or a polycarboxylic acid. Other subjects of the present invention are an optical waveguide manufactured using the cladding material and a method for manufacturing the optical waveguide.

The cladding material preferably further contains a carbon nanotube. The carbon nanotube is dispersed in the polymer compound containing an oxazoline structure in a side chain as a matrix material, whereby the cladding material can make the resistance value of a cladding considerably lower than the resistance value of a core and achieves an optical waveguide modulator that is low in applied voltage for electric field orientation and is considerably low in optical modulation operating voltage.

The following describes the present invention in more detail.

[Cladding Material]

<Polymer Compound Containing Oxazoline Structure in Side Chain>

The polymer material used as the cladding material according to the present invention is a polymer having an oxazoline structure in a side chain. In the cladding material containing the carbon nanotube, the polymer compound also plays the role of a polymer matrix that disperses the carbon nanotube.

In the present invention, the polymer having an oxazoline structure in a side chain (hereinafter, referred to as a oxazoline polymer) is not limited to a particular polymer so long as it is a polymer in which an oxazoline group bonds to a repeating unit forming a main chain directly or via a spacer group such as an alkylene group and is specifically preferably a polymer having a repeating unit that bonds to the polymer main chain or the spacer group at the 2-position of an oxazoline ring obtained by radically polymerizing an oxazoline monomer having a polymerizable carbon-carbon double bond-containing group at the 2-position of an oxazoline ring of Formula [1] below.

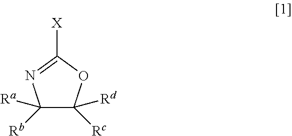

In the formula, X is a polymerizable carbon-carbon double bond-containing group, R^{a} to R^{d} are mutually independently a hydrogen atom, a halogen atom, linear or branched C₁₋₅ alkyl groups, a C₆₋₂₀ aryl group, or a C₇₋₂₀ aralkyl group.

The polymerizable carbon-carbon double bond-containing group of the oxazoline monomer is not limited to a particular group so long as it contains a polymerizable carbon-carbon double bond; preferable examples thereof include chain hydrocarbon groups containing the polymerizable carbon-carbon double bond, in which preferable examples include a C₂₋₈ alkenyl group such as vinyl group, allyl group, and isopropenyl group.

Examples of the halogen atom include a fluorine atom, a chlorine atom, a bromine atom, and an iodine atom.

Specific examples of the linear or branched C₁₋₅ alkyl groups include methyl group, ethyl group, n-propyl group, isopropyl group, n-butyl group, sec-butyl group, tert-butyl group, and n-pentyl group.

Specific examples of the C₆₋₂₀ aryl group include phenyl group, xylyl group, tolyl group, biphenyl group, and naphthyl group.

Specific examples of the C₇₋₂₀ aralkyl group include benzyl group, phenylethyl group, and phenylcyclohexyl group.

Specific examples of the oxazoline monomer having the polymerizable carbon-carbon double bond-containing group at the 2-position of the oxazoline ring of Formula [1] include 2-vinyl-2-oxazoline, 2-vinyl-4-methyl-2-oxazoline, 2-vinyl-4-ethyl-2-oxazoline, 2-vinyl-4-propyl-2-oxazoline, 2-vinyl-4-butyl-2-oxazoline, 2-vinyl-5-methyl-2-oxazoline, 2-vinyl-5-ethyl-2-oxazoline, 2-vinyl-5-propyl-2-oxazoline, 2-vinyl-5-butyl-2-oxazoline, 2-isopropenyl-2-oxazoline, 2-isopropenyl-4-methyl-2-oxazoline, 2-isopropenyl-4-ethyl-2-oxazoline, 2-isopropenyl-4-propyl-2-oxazoline, 2-isopropenyl-4-butyl-2-oxazoline, 2-isopropenyl-5-methyl-2-oxazoline, 2-isopropenyl-5-ethyl-2-oxazoline, 2-isopropenyl-5-propyl-2-oxazoline, and 2-isopropenyl-5-butyl-2-oxazoline; among them, 2-isopropenyl-2-oxazoline is preferred in view of availability and the like.

Considering that materials using water as a solvent or a preparation solvent have been demanded in recent years from a tendency to get rid of organic solvents, assuming that the cladding material of the present invention is prepared in an aqueous system, the oxazoline polymer is preferably water-soluble.

Such a water-soluble oxazoline polymer may be a homopolymer of the oxazoline monomer of Formula [1], but is preferably a polymer obtained by radically polymerizing at least two kinds of monomers of the oxazoline monomer and a hydrophilic functional group-containing (meth)acrylic monomer in order to further increase solubility in water. In the present invention, the (meth)acrylic monomer refers to (meth)acrylic acid and (meth) acrylate, and the wording “(meth)acrylic acid” means both acrylic acid and methacrylic acid.

Specific examples of the hydrophilic functional group-containing (meth)acrylic monomer include (meth)acrylic acid, 2-hydroxyethyl (meth)acrylate, polyethylene glycol monomethyl ether (meth)acrylate, polyethylene glycol mono(meth)acrylate, 2-aminoethyl (meth)acrylate and salts thereof, sodium (meth)acrylate, ammonium (meth)acrylate, (meth)acrylonitrile, (meth)acrylamide, N-methylol(meth)acrylamide, and N-(2-hydroxyethyl)(meth)acrylamide; each of these may be used singly, or two or more of them may be used in combination. Among these, preferred ones are polyethylene glycol monomethyl ether (meth)acrylate and polyethylene glycol mono(meth)acrylate.

In the present invention, other monomers apart from the oxazoline monomer and the hydrophilic functional group-containing (meth)acrylic monomer can be used in combination.

Specific examples of the other monomers include (meth)acrylate monomers such as methyl (meth)acrylate, ethyl (meth)acrylate, butyl (meth)acrylate, 2-ethylhexyl (meth)acrylate, stearyl (meth)acrylate, perfluoroethyl (meth)acrylate, and phenyl (meth)acrylate; a-olefinic monomers such as ethylene, propylene, butene, and pentene; haloolefinic monomers such as vinyl chloride, vinylidene chloride, and vinyl fluoride; styrenic monomers such as styrene and a-methyl styrene; vinyl carboxylate monomers such as vinyl acetate and vinyl propionate; and vinyl ether monomers such as methyl vinyl ether and ethyl vinyl ether; each of these may be used singly, or two or more of them may be used in combination.

The content of the oxazoline monomer in the monomer components used in the manufacture of the oxazoline polymer used in the present invention is preferably 10% by mass or more, more preferably 20% by mass or more, and further preferably 30% by mass or more. The upper limit value of the content of the oxazoline monomer in the monomer components is 100% by mass; in this case, the homopolymer of the oxazoline monomer is obtained.

In view of increasing the water-solubility of the oxazoline polymer to be obtained, the content of the hydrophilic functional group-containing (meth)acrylic monomer in the monomer components is preferably 10% by mass or more, more preferably 20% by mass or more, and further preferably 30% by mass or more.

The content of the other monomers in the monomer components, which varies by their types and cannot be unconditionally determined, may be set as appropriate in the range of 5% by mass to 95% by mass or less and preferably 10% by mass to 90% by mass or less.

The average molecular weight of the oxazoline polymer is not limited to a particular molecular weight; the weight average molecular weight thereof is preferably 1,000 to 2,000,000. The oxazoline polymer more preferably has a weight average molecular weight of 2,000 to 1,000,000.

The weight average molecular weights in the present invention are measured values (in terms of polystyrene) by gel permeation chromatography.

The oxazoline polymer used in the present invention can be manufactured by polymerizing the various kinds of monomers by a known radical polymerization described in Japanese Patent Application Publication No. H06-32844 or Japanese Patent Application Publication No. 2013-72002, for example.

The oxazoline polymer that can be used in the present invention can also be obtained as commercially available products; examples of the commercially available products include Epocros (registered trademark) WS-300 (an aqueous solution with a solid content of 10% by mass), Epocros WS-700 (an aqueous solution with a solid content of 25% by mass), and Epocros WS-500 (a solution of water/1-methoxy-2-propanol with a solid content of 39% by mass) [manufactured by Nippon Shokubai Co., Ltd.]; and poly(2-isopropenyl-2-oxazoline-co-methyl methacrylate) [manufactured by Aldrich].

When the oxazoline polymer is commercially available as a solution, it may be used as it is to be the cladding material or may be subjected to solvent substitution to be a target solvent-based cladding material.

<Acid Generator>

The cladding material of the present invention contains the acid generator in addition to the oxazoline polymer.

The acid generator is a compound that ring-opening polymerizes the oxazoline group of the oxazoline polymer, in other words, plays the role of a polymerization initiator, and can increase the solvent resistance of a hardened film and the like formed using the cladding material of the present invention.

The acid generator is not limited so long as it is a substance that generates an acid by external stimuli such as light and/or heat and may be a high molecular compound or a low molecular compound.

A photoacid generator that generates cations by light may be selected from known ones as appropriate; examples thereof include onium salt derivatives such as diazonium salts, sulfonium salts, and iodonium salts.

Specific examples thereof include aryl diazonium salts such as phenyldiazonium hexafluorophosphate, 4-methoxyphenyldiazonium hexafluoroantimonate, and 4-methylphenyldiazonium hexafluorophosphate; diaryliodonium salts such as diphenyliodonium hexafluoroantimonate, di(4-methylphenyl)iodonium hexafluorophosphate, and di(4-tert-butylphenyl)iodonium hexafluorophosphate; and triarylsulfonium salts such as triphenylsulfonium hexafluoroantimonate, tris(4-methoxyphenyl)sulfonium hexafluorophosphate, diphenyl-4-thiophenoxyphenylsulfonium hexafluoroantimonate, diphenyl-4-thiophenoxyphenylsulfonium hexafluorophosphate, 4,4′-bis(diphenylsulfonio)phenylsulfide-bishexafluoroantimonate, 4,4′-bis(diphenylsulfonio)phenylsulfide-bishexafluorophosphate, 4,4′-bis[di(β-hydroxyethoxy)phenylsulfonio]phenylsulfide-bishexafluoroantimonate, 4,4-bis [di(β-hydroxyethoxy)phenylsulfonio]phenylsulfide-bishexafluorophosphate, 4-[4′-(benzoyl)phenylthio]phenyl-di(4-fluorophenyl)sulfonium hexafluoroantimonate, and 4-[4′-(benzoyl)phenylthio]phenyl-di(4-fluorophenyl)sulfonium hexafluorophosphate.

These onium salts may be commercially available products; specific examples thereof include San-Aid SI-60, SI-80, SI-100, SI-60L, SI-80L, SI-100L, SI-L145, SI-L150, SI-L160, SI-L110, and SI-L147 [manufactured by Sanshin Chemical Industry Co., Ltd.]; UVI-6950, UVI-6970, UVI-6974, UVI-6990, and UVI-6992 [manufactured by Union Carbide Corporation]; CPI-100P, CPI-100A, CPI-101A, CPI-200K, and CPI-200S [manufactured by San-Apro Ltd]; Adekaoptomer SP-150, SP-151, SP-170, and SP-171 [manufactured by Adeka Corporation]; Irgacure 261 [manufactured by BASF]; CI-2481, CI-2624, CI-2639, and CI-2064 [manufactured by Nippon Soda Co., Ltd.]; CD-1010, CD-1011, and CD-1012 [manufactured by Sartomer]; DS-100, DS-101, DAM-101, DAM-102, DAM-105, DAM-201, DSM-301, NAI-100, NAI-101, NAI-105, NAI-106, SI-100, SI-101, SI-105, SI-106, PI-105, NDI-105, BENZOIN TOSYLATE, MBZ-101, MBZ-301, PYR-100, PYR-200, DNB-101, NB-101, NB-201, BBI-101, BBI-102, BBI-103, and BBI-109 [manufactured by Midori Kagaku Co., Ltd.]; PCI-061T, PCI-062T, PCI-020T, and PCI-022T [manufactured by Nippon Kayaku Co., Ltd.]; IBPF and IBCF [manufactured by Sanwa Chemical Co., Ltd.]; and PI2074 [manufactured by Rhodia Japan, Ltd.].

Each of the photoacid generators described above may be used singly, or two or more of them may be used in combination.

A thermal acid generator that generates cations by heat may be selected from known ones as appropriate; examples thereof include triaryl sulfonium salts, dialkyl aryl sulfonium salts, and diaryl alkyl sulfonium salts of strong non-nucleophilic acids; alkyl aryl iodonium salts and diaryl iodonium salts of strong non-nucleophilic acids; and ammonium, alkylammonium, dialkylammonium, trialkylammonium, and tetraalkylammonium salts of strong non-nucleophilic acids.

Covalent thermal acid generators can also be used; examples thereof include 2-nitrobenzylester of alkyl or aryl sulfonic acids and other esters of sulfonic acid that decompose by heat to give free sulfonic acid.

Specific Examples thereof include diaryl iodonium perfluoroalkyl sulfonate, diaryl iodonium tris(fluoroalkylsulfonyl) methide, diaryl iodonium bis(fluoroalkylsulfonyl) methide, diaryl iodonium bis(fluoroalkylsulfonyl) imide, and diaryl iodonium quaternary ammonium perfluoroalkyl sulfonate; benzene tosylates such as 2-nitrobenzyl tosylate, 2,4-dinitrobenzyl tosylate, 2,6-dinitrobenzyl tosylate, and 4-nitrobenzyl tosylate; benzene sulfonates such as p-toluenesulfonic acid cyclohexyl, 2-trifluoromethyl-6-nitrobenzyl 4-chlorobenzenesulfonate, and 2-trifluoromethyl-6-nitrobenzyl 4-nitrobenzenesulfonate; phenolic sulfonate esters such as phenyl 4-methoxybenzenesulfonate; quaternary ammonium tris(fluoroalkylsulfonyl) methide; quaternary alkylammonium bis(fluoroalkylsulfonyl) imide; and alkylammonium salts of organic acids such as the triethylammonium salt of 10-camphorsulfonic acid.

Further, amine salts of various aromatic (anthracene, naphthalene, or benzene derivatives) sulfonic acids can also be used; specific examples thereof include amine salts of sulfonic acids described in specifications of U.S. Pat. No. 3,474,054, U.S. Pat. No. 4,200,729, U.S. Pat. No. 4,251,665, and U.S. Pat. No. 5,187,019.

Each of the thermal acid generators described above may be used singly, or two or more of them may be used in combination.

<Polycarboxylic Acid>

The cladding material of the present invention contains the polycarboxylic acid in addition to the oxazoline polymer.

The polycarboxylic acid is a compound that causes a cross-linking reaction with the oxazoline group of the oxazoline polymer, in other words, plays the role of a cross-linking agent, and can increase the solvent resistance of the hardened film and the like formed using the cladding material of the present invention.

The polycarboxylic acid is not limited to a particular compound so long as it is a compound having two or more carboxy groups as functional groups having reactivity with the oxazoline group, and the compound may further have functional groups having reactivity with the oxazoline group such as thiol group, amino group, sulfinic acid group, and epoxy group in addition to the carboxy groups.

Among them, preferred examples are polycarboxylic acids with a molecular weight of 1,000 or lower; examples thereof include aliphatic dicarboxylic acids such as oxalic acid, malonic acid, succinic acid, glutaric acid, adipic acid, piperic acid, suberic acid, azelaic acid, and sebacic acid; aliphatic unsaturated carboxylic acids such as maleic acid and fumaric acid; aromatic dicarboxylic acids such as phthalic acid, isophthalic acid, and terephthalic acid, (meth)acrylic acid, and (meth)acrylic acid oligomers. Among them, hydroxycarboxylic acids are particularly preferred; examples thereof include aliphatic oxyacids such as glycolic acid, lactic acid, hydroxy(alkyl)acrylic acids, α-oxylactic acid, glyceric acid, tartronic acid, malic acid, tartaric acid, and citric acid; and aromatic oxyacids such as salicylic acid, oxybenzoic acid, gallic acid, mandelic acid, and tropic acid. One or a mixture of two or more selected from these groups can be used; the most preferred one is citric acid.

<Carbon Nanotube>

The carbon nanotube (hereinafter, also referred to as CNT) contained in the cladding material of the present invention is generally manufactured by arc discharge, chemical vapor deposition (CVD), laser abrasion, or the like; CNT used in the present invention may be obtained by any method. CNT includes a single-walled CNT (hereinafter, denoted as SWCNT) in which one carbon film (graphene sheet) is wound in a cylindrical shape, a double-walled CNT (hereinafter, denoted as DWCNT) in which two graphene sheets are concentrically wound, and a multi-walled CNT (hereinafter, denoted as MWCNT) in which a plurality of graphene sheets are concentrically wound; in the present invention, each of SWCNT, DWCNT, and MWCNT can be used singly, or two or more of them can be used in combination.

When SWCNT, DWCNT, or MWCNT is manufactured by the above methods, fullerene, graphite, or amorphous carbon may be simultaneously generated as byproducts, or catalytic metals such as nickel, iron, cobalt, and yttrium may remain in the product, and the removal of these impurities and/or refining may be required. For the removal of the impurities, as well as acid treatment with nitric acid, sulfuric acid, or the like, ultrasonic treatment is effective. However, the acid treatment with nitric acid, sulfuric acid, or the like may break a π-conjugated system forming CNT to impair characteristics intrinsic to CNT, and it is preferred that CNT be refined on appropriate conditions to be used.

When the cladding material of the present invention contains the carbon nanotube, the carbon nanotube is dispersed in the polymer compound containing an oxazoline structure in a side chain as a matrix material.

However, the carbon nanotube generally has a problem in that it is difficult to be dispersed, and to increase the dispersability, the carbon nanotube may be used as a carbon nanotube dispersion liquid with a carbon nanotube dispersant (a CNT dispersant) used in combination. A modified carbon nanotube with the carbon nanotube modified with various kinds of functional groups may be used.

Examples of the modified carbon nanotube include a polyethylene glycol-modified carbon nanotube, a polyaminobenzene sulfonic acid-modified carbon nanotube, a carboxylic acid-modified carbon nanotube, an octadecylamine-modified carbon nanotube, and an amide-modified carbon nanotube. Among them, assuming that the cladding material of the present invention is prepared in an aqueous system as described above, preferred ones are the polyethylene glycol-modified carbon nanotube and the polyaminobenzene sulfonic acid-modified carbon nanotube, which are excellent in solubility and dispersability in water, and a particularly preferred one is the polyethylene glycol-modified carbon nanotube.

As to the CNT dispersant, conventionally known ones can be used as appropriate. Among them, a highly branched polymer described in WO 2012/161307 can be suitably used as the CNT dispersant.

Specific examples thereof include a highly branched polymer having a repeating unit of Formula [5] or Formula [6] below:

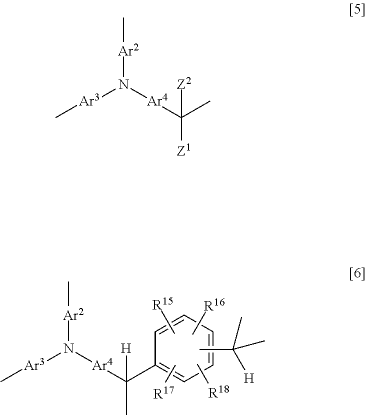

[in Formulae [5] and [6], Ar² to Ar⁴ are each independently any divalent organic group of Formulae [7] to [11]; Z¹ and Z² are each independently a hydrogen atom, a C₁₋₅ alkyl group optionally having a branched structure, or any monovalent organic group of Formulae [12] to [15] (where Z¹ and Z² are not simultaneously the alkyl group), in Formula [6], R¹⁵ to R¹⁸ are each independently a hydrogen atom, a halogen atom, a C₁₋₅ alkyl group optionally having a branched structure, a C₁₋₅ alkoxy group optionally having a branched structure, a carboxy group, a sulfo group, a phosphoric acid group, a phosphonic acid group, or salts thereof:

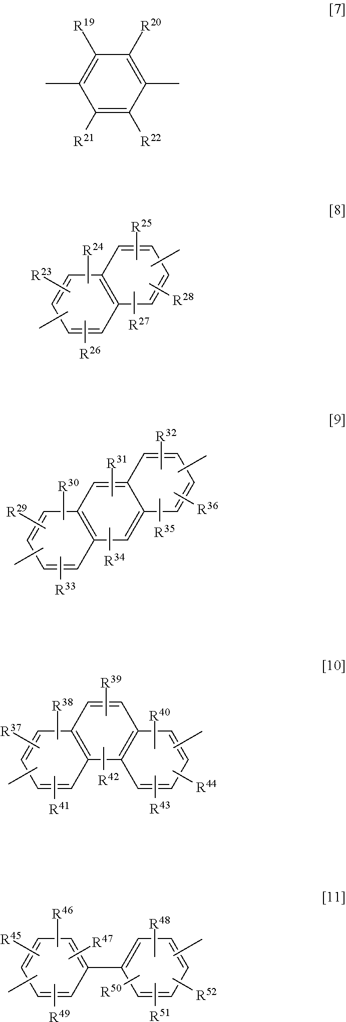

(in the formulae, R¹⁹ to R⁵² are each independently a hydrogen atom, a halogen atom, a C₁₋₅ alkyl group optionally having a branched structure, a C₁₋₅ alkoxy group optionally having a branched structure, a carboxy group, a sulfo group, a phosphoric acid group, a phosphonic acid group, or salts thereof)

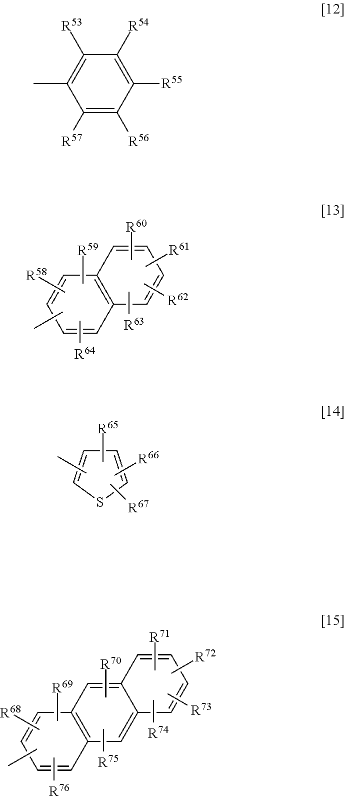

{in the formulae, R⁵³ to R⁷⁶ are each independently a hydrogen atom, a halogen atom, a C₁₋₅ alkyl group optionally having a branched structure, a C₁₋₅ haloalkyl group optionally having a branched structure, a phenyl group, OR⁷⁷, COR⁷⁷, NR⁷⁷R⁷⁸, COOR⁷⁹ (in these formulae, R⁷⁷ and R⁷⁸ are each independently a hydrogen atom, a C₁₋₅ alkyl group optionally having a branched structure, a C₁₋₅ haloalkyl group optionally having a branched structure, or a phenyl group; and R⁷⁹ is a C₁₋₅ alkyl group optionally having a branched structure, a C₁₋₅ haloalkyl group optionally having a branched structure, or a phenyl group), a carboxy group, a sulfo group, a phosphoric acid group, a phosphonic acid group, or salts thereof}

where at least one aromatic ring of the repeating unit of Formula [5] or Formula [6] has at least one acidic group selected from a carboxy group, a sulfo group, a phosphoric acid group, a phosphonic acid group, and salts thereof].

In Formulae [5] and [6], Ar² to Ar⁴ are each independently any divalent organic group of Formulae [7] to [11]; a particularly preferred one is a substituted or unsubstituted phenylene group of Formula [7].

In R¹⁵ to R⁵² in Formulae [6] to [11], examples of the halogen atom include a fluorine atom, a chlorine atom, a bromine atom, and iodine atom.

Examples of the C₁₋₅ alkyl group optionally having a branched structure include methyl group, ethyl group, n-propyl group, isopropyl group, n-butyl group, sec-butyl group, tert-butyl group, and n-pentyl group.

Examples of the C₁₋₅ alkoxy group optionally having a branched structure include methoxy group, ethoxy group, n-propoxy group, isopropoxy group, n-butoxy group, sec-butoxy group, tert-butoxy group, and n-pentoxy group.

Examples of the salts of carboxy group, sulfo group, phosphoric acid group, and phosphonic acid group include alkali metallic salts of sodium, potassium, and the like;

alkaline earth metallic salts of magnesium, calcium, and the like; ammonium salts; aliphatic amine salts of C₁₋₁₀ trialkylamines such as propylamine, dimethylamine, triethylamine, tri-n-propylamine, tri-n-butylamine, tri-n-pentylamine, tri-n-hexylamine, tri-n-heptylamine, tri-n-octylamine, tri-n-nonylamine, and tri-n-decylamine, ethylenediamine, and the like; cyclic amine salts of imidazolin, piperazine, morpholine, and the like; aromatic amine salts of aniline, diphenylamine, and the like; and pyridinium salts.

In Formulae [5] and [6], Z¹ and Z² are preferably each independently a hydrogen atom, a 2- or 3-thienyl group, or the group of Formula [12], in particular, more preferably either Z¹ or Z² is a hydrogen atom, and the other is a hydrogen atom, a 2- or 3-thienyl group, or the group of Formula [12], in which R⁵⁵ is a phenyl group or R⁵⁵ is a methoxy group in particular.

When R⁵⁵ is a phenyl group, when a technique that introduces an acidic group after manufacturing the polymer is used in an acidic group introduction process described below, the acidic group may be introduced on this phenyl group.

In Z¹ and Z², examples of the C₁₋₅ alkyl group optionally having a branched structure include ones similar to those exemplified in Formulae [6] to [11].

In Formulae [12] to [15], examples of the C₁₋₅ haloalkyl group optionally having a branched structure in R⁵³ to R⁷⁶ include difluoromethyl group, trifluoromethyl group, bromodifluoromethyl group, 2-chloroethyl group, 2-bromoethyl group, 1,1-difluoroethyl group, 2,2,2-trifluoroethyl group, 1,1,2,2-tetrafluoroethyl group, 2-chloro-1,1,2-trifluoroethyl group, pentafluoroethyl group, 3-bromopropyl group, 2,2,3,3-tetrafluoropropyl group, 1,1,2,3,3,3-hexafluoropropyl group, 1,1,1,3,3,3-hexafluoropropan-2-yl group, 3-bromo-2-methylpropyl group, 4-bromobutyl group, and perfluoropentyl group.

Examples of the halogen atom, the C₁₋₅ alkyl group optionally having a branched structure, and the salts of carboxy group, sulfo group, phosphoric acid group, and phosphoric acid group include ones similar to those exemplified in Formulae [6] to [11].

Among the highly branched polymers having the repeating unit of Formula [5] or [6], a preferred one is a highly branched polymer the repeating unit of which is Formula [16]:

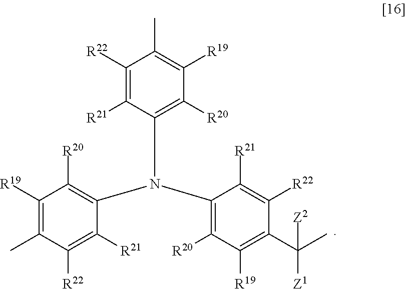

(where R′⁹ to R²² are a hydrogen atom, a carboxy group, a sulfo group, a phosphoric acid group, a phosphonic acid group, or salts thereof; and Z¹ and Z² represent the same meaning as the above).

Among them, a highly branched polymer having a repeating unit having an acidic group such as sulfo group of Formula [17] below, for example, is suitable as the CNT dispersant:

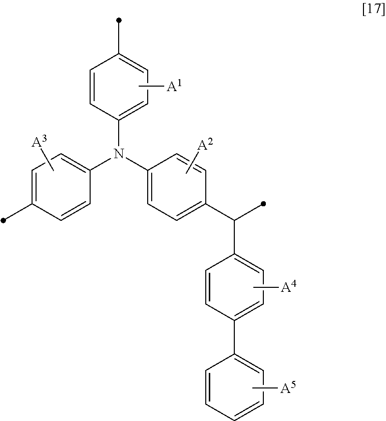

(where any one of A¹ to A⁵ is a sulfo group; the others are hydrogen atoms; and black dots indicate bonding ends).

Although the average molecular weight of the highly branched polymer is not limited to a particular value, a weight average molecular weight represented by a measured value (in terms of polystyrene) by gel permeation chromatography is preferably 1,000 to 2,000,000. If the weight average molecular weight of the polymer is less than 1,000, the dispersability of CNT may markedly decrease, or the dispersability may fail to be exhibited. In contrast, if the weight average molecular weight exceeds 2,000,000, handling during dispersion treatment may be extremely difficult. The highly branched polymer with a weight average molecular weight of 2,000 to 1,000,000 is more preferred.

The highly branched polymer having the repeating unit of Formula [5] or [6] is a polymer containing a triarylamine structure as a branching point, or more specifically, a polymer obtained by condensation polymerizing triarylamines with aldehydes and/or ketones in an acidic condition.

It is considered that this highly branched polymer shows high affinity for the conjugated structure of CNT through π-π interaction originating from the aromatic rings of the triarylamine structure, and high CNT dispersability appears. In addition, this highly branched polymer, having the branched structure, also has high solubility that is not observed in a linear one and is also excellent in thermal stability.

Examples of the aldehyde compound for use in the manufacture of the highly branched polymer include saturated aliphatic aldehydes such as formaldehyde, paraformaldehyde, acetaldehyde, propyl aldehyde, butyl aldehyde, isobutyl aldehyde, valeraldehyde, capronaldehyde, 2-methylbutyl aldehyde, hexyl aldehyde, undecane aldehyde, 7-methoxy-3,7-dimethyloctyl aldehyde, cyclohexane aldehyde, 3-methyl-2-butyl aldehyde, glyoxal, malonaldehyde, succinaldehyde, glutaraldehyde, and adipaldehyde; unsaturated aliphatic aldehydes such as acrolein and methacrolein; heterocyclic aldehydes such as furfural, pyridine aldehyde, and thiophene aldehyde; and aromatic aldehydes such as benzaldehyde, tolyl aldehyde, trifluoromethylbenzaldehyde, phenylbenzaldehyde, salicylaldehyde, anisaldehyde, acetoxybenzaldehyde, terephthalaldehyde, acetylbenzaldehyde, formylbenzoic acid, methyl formylbenzoate, aminobenzaldehyde, N,N-dimethylaminobenzaldehyde, N,N-diphenylaminobenzaldehyde, naphthylaldehyde, anthrylaldehyde, phenanthrylaldehyde, phenylacetaldehyde, and 3-phenylpropionaldehyde. Aromatic aldehydes are particularly preferred.

The ketone compound for use in the manufacture of the highly branched polymer is alkyl aryl ketones or diaryl ketones; examples thereof include acetophenone, propiophenone, diphenyl ketone, phenyl naphthyl ketone, dinaphthyl ketone, phenyl tolyl ketone, and ditolyl ketone.

The mixing ratio between the highly branched polymer (the dispersant) and CNT can be about 1,000:1 to 1:100 in terms of mass ratio.

<Solvent>

The cladding material of the present invention may further contain a solvent. The solvent is not limited to a particular solvent so long as it can dissolve and/or disperse the polymer compound containing an oxazoline structure in a side chain, the acid generator or the polycarboxylic acid, and, as needed, the carbon nanotube and other components described below; examples thereof include organic solvents having dissolving ability for the highly branched polymer (the CNT dispersant), a mixed solvent of a hydrophilic solvent among the organic solvents and water, and a solvent of water alone.

Specific examples of the solvent include water and organic solvents such as ethers such as tetrahydrofuran (THF), diethyl ether, and 1,2-dimethoxyethane (DME); halogenated hydrocarbons such as methylene chloride, chloroform, and 1,2-dichloroethane; amides such as N,N-dimethylformamide (DMF), N,N-dimethylacetamide (DMAc), and N-methyl-2-pyrrolidone (NMP); ketones such as acetone, methyl ethyl ketone, methyl isobutyl ketone, and cyclohexanone; alcohols such as methanol, ethanol, 2-propanol, and n-propanol; aliphatic hydrocarbons such as n-heptane, n-hexane, and cyclohexane; aromatic hydrocarbons such as benzene, toluene, xylene, and ethylbenzene; glycol ethers such as ethylene glycol monoethyl ether, ethylene glycol monobutyl ether, and propylene glycol monomethyl ether; and glycols such as ethylene glycol and propylene glycol; each of these solvents can be used singly, or two or more of them can be used in a mixed manner. In particular, in view of the possibility of improving the rate of the isolated dispersion of the carbon nanotube, preferred ones are water, NMP, DMF, THF, methanol, and 2-propanol.

In recent years, materials using water as a solvent have been demanded from a tendency to get rid of organic solvents, and it is preferred that a mixed solvent of a hydrophilic solvent such as alcohols, glycols, or glycol ethers and water or a solvent of water alone be used also in the cladding material liquid of the present invention.

<Other Containable Components>

The cladding material of the present invention can contain cross-linking agents, surfactants, leveling agents, antioxidants, optical stabilizers, and the like as appropriate to the extent that the performance as the cladding material of an optical waveguide is not affected.

<Preparation of Cladding Material>

A method for preparing the cladding material of the present invention is any method, and the cladding material can be prepared by mixing the oxazoline polymer, the acid generator or the polycarboxylic acid, and, as needed, the carbon nanotube (or the CNT dispersion liquid with the carbon nanotube dispersed with the CNT dispersant), the solvent, and the other possible components in any order.

When the cladding material containing the carbon nanotube is prepared, a mixture containing the oxazoline polymer, the carbon nanotube (or the CNT dispersion liquid), the acid generator or the polycarboxylic acid, and, as needed, the solvent and the like is preferably subjected to dispersion treatment, and this treatment can further increase the dispersion rate of the carbon nanotube. Examples of the dispersion treatment include wet treatment using a ball mill, a bead mill, a jet mill or the like as mechanical treatment and ultrasonic treatment using a bath type or probe type sonicator.

Although the time for the dispersion treatment may be any amount, the time is preferably about 1 minute to 10 hours.

The oxazoline polymer for use in the present invention is excellent in the dispersability of the carbon nanotube, and even when heat treatment is not performed before the dispersion treatment or the like, a composition with the carbon nanotube dispersed in a high concentration can be obtained; however, the heat treatment may be performed as needed.

In the cladding material of the present invention, the amount to be added of the carbon nanotube relative to 100 parts by mass of the oxazoline polymer is 0.00001 part by mass to 10 parts by mass, for example, preferably 0.00005 part by mass to 5 parts by mass, and more preferably 0.0001 part by mass to 1 part by mass.

In the cladding material of the present invention, the amount to be added of the acid generator or the polycarboxylic acid relative to 100 parts by mass of the oxazoline polymer, which is not limited to a particular amount because it also depends on the content of the oxazoline group in the oxazoline polymer, is 0.0001 part by mass to 20 parts by mass, for example, preferably 0.0005 part by mass to 10 parts by mass, and more preferably 0.001 part by mass to 3 parts by mass.

When the cladding material of the present invention is dissolved and/or dispersed in the solvent to make varnish, its solid content is 1% by mass to 80% by mass, for example, preferably 10% by mass to 50% by mass, and more preferably 15 parts by mass to 35 parts by mass. The solid content indicates the entire components except the solvent.

[Optical Waveguide]

The optical waveguide of the present invention is an optical waveguide including a core and a cladding that surrounds the entire outer periphery of the core and has a refractive index lower than that of the core, the cladding being formed of a cladding material containing the polymer compound containing an oxazoline structure in a side chain and the acid generator or the polycarboxylic acid.

<Core>

In the optical waveguide of the present invention, the core may be formed of a material having a refractive index higher than the refractive index of the formed cladding.

The core preferably contains an organic nonlinear optical compound exhibiting a second-order nonlinear optical effect in the form of being dispersed in a polymer matrix or contains the organic nonlinear optical compound in the form of bonding to a side chain of a polymer compound, for example. The organic nonlinear optical compound is preferably a nonlinear optical compound having a tricyano-bonded furan ring of Formula [2], for example.

In Formula [2], the C₁₋₁₀ alkyl group in R′ and R² may have a branched structure or a cyclic structure or may be an arylalkyl group; examples thereof include methyl group, ethyl group, n-propyl group, isopropyl group, cyclopropyl group, n-butyl group, isobutyl group, sec-butyl group, tert-butyl group, n-pentyl group, neopentyl group, cyclopentyl group, n-hexyl group, cyclohexyl group, n-octyl group, n-decyl group, 1-adamantyl group, benzyl group, and phenethyl group.

Examples of the C₆₋₁₀ aryl group include phenyl group, tolyl group, xylyl group, and naphthyl group.

Examples of the substituent that the C₁₋₁₀ alkyl group and the C₆₋₁₀ aryl group optionally have include amino group; hydroxy group; alkoxycarbonyl groups such as methoxycarbonyl group and tert-butoxycarbonyl group; silyloxy groups such as trimethylsilyloxy group, tert-butyldimethylsilyloxy group, tert-butyldiphenylsilyloxy group, and triphenylsilyloxy group; and halogen atoms.

Examples of the C₁₋₁₀ alkyl group in R³ to R⁶ include the same ones as the above.

The C₁₋₁₀ alkoxy group may have a branched structure or a cyclic structure or may be an arylalkyloxy group; examples thereof include methoxy group, ethoxy group, n-propoxy group, isopropoxy group, cyclopropoxy group, n-butoxy group, isobutoxy group, sec-butoxy group, tert-butoxy group, n-pentyloxy group, neopentyloxy group, cyclopentyloxy group, n-hexyloxy group, cyclohexyloxy group, n-octyloxy group, n-decyloxy group, 1-adamantyloxy group, benzyloxy group, and phenetoxy group.

The C₂₋₁₁ alkylcarbonyloxy group may have a branched structure or a cyclic structure or may be an arylalkylcarbonyloxy group; examples thereof include acetoxy group, propionyloxy group, butylyloxy group, isobutylyloxy group, cyclopropanecarbonyloxy group, pentanoyloxy group, 2-methylbutanoyloxy group, 3-methylbutanoyloxy group, pivaloyloxy group, hexanoyloxy group, 3,3-dimethylbutanoyloxy group, cyclopentanecarbonyloxy group, heptanoyloxy group, cyclohexanecarbonyloxy group, n-nonanoyloxy group, n-undecanoyloxy group, 1-adamantanecarbonyloxy group, phenylacetoxy group, and 3-phenylpropanoyloxy group.

Examples of the C₄₋₁₀ aryloxy group include phenoxy group, naphthalen-2-yloxy group, furan-3-yloxy group, and thiophen-2-yloxy group.

Examples of the C₅₋₁₁ arylcarbonyloxy group include benzoyloxy group, 1-naphthoyloxy group, furan-2-carbonyloxy group, and thiophene-3-carbonyloxy group.

Examples of the silyloxy group having the C₁₋₆ alkyl group and/or phenyl group include silyloxy groups such as trimethylsilyloxy group, tert-butyldimethylsilyloxy group, tert-butyldiphenylsilyloxy group, and triphenylsilyloxy group.

Examples of the halogen atom include the same ones as exemplified for R¹⁵ to R⁵².

Examples of the C₁₋₅ alkyl group in R⁷ and R⁸ include the same ones as exemplified for R¹⁵ to R⁵².

Examples of the C₁₋₅ haloalkyl group include the same ones as exemplified for R⁵³ to R⁷⁶.

Examples of the C₆₋₁₀ aryl group include the same ones as exemplified for R¹ and

R².

Specific combinations of R⁷ and R⁸ are preferably methyl group-methyl group, methyl group-trifluoromethyl group, and trifluoromethyl group-phenyl group.

In Formulae [3] and [4], specific examples of the C₁₋₁₀ alkyl group, the C₆₋₁₀ aryl group, and the substituent in R⁹ to R¹⁴ include the ones exemplified above.

As a compound corresponding to the nonlinear optical compound for use in the present invention, as a nonlinear optical compound having a developed π-conjugated chain and a tricyano heterocyclic structure, which is an extremely strong electron withdrawing group, and having an extremely strong molecular hyperpolarizability β, the following compound is reported (Chem. Mater. 2001, 13, 3043-3050).

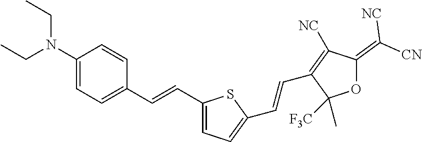 [SMILES: CCN(CC)c1ccc(/C=C/c2ccc(/C=C/C3=C(C#N)C(=C(C#N)C#N)OC3(C)C(F)(F)F)s2)cc1]

Further, a dialkylanilino moiety as an electron donating group in the structure is changed to various structures, whereby the molecular hyperpolarizability can be further increased (J. Polym. Sci. Part A. 2011, Vol. 49, p.4′7).

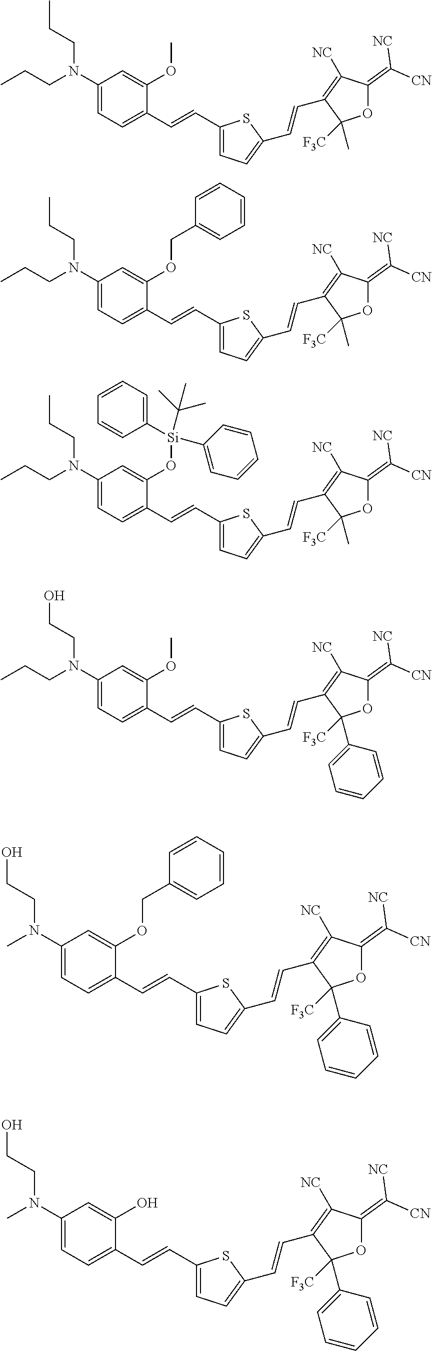

When the nonlinear optical compound is dispersed in the polymer matrix, the nonlinear optical compound is required to be dispersed in a high concentration and uniformly in the matrix, and the polymer matrix preferably exhibits high compatibility with the nonlinear optical compound. In view of being used as the core of the optical waveguide, the polymer matrix preferably has excellent transparency and moldability.

Examples of such a polymer matrix material include resins such as poly(methyl methacrylate), polycarbonate, polystyrene, silicone resins, epoxy resins, polysulfone, polyethersulfone, and polyimide.

Examples of a technique for dispersing the nonlinear optical compound in the polymer matrix include a method that dissolves the nonlinear optical compound and the matrix material in an organic solvent or the like with an appropriate ratio, applies the resultant mixture to a substrate, and dries the applied mixture to form a thin film (a hardened film).

When the nonlinear optical compound is bonded to the side chain of the polymer compound, the side chain of the polymer compound is required to have a functional group that can form a covalent bond with the nonlinear optical compound; examples of the functional group include isocyanate group, hydroxy group, carboxy group, epoxy group, amino group, halogenated allyl groups, and halogenated acyl groups. These functional groups can form a covalent bond with the hydroxy group or the like of the nonlinear optical compound having the tricyano-bonded furan ring of Formula [2].

When the nonlinear optical compound is bonded to the side chain of the polymer compound, to adjust the content of the nonlinear optical compound, the core may be in a form in which the unit structure of the polymer matrix and the unit structure of the polymer compound to which the nonlinear optical compound is bonded are copolymerized in a sense.

The proportion of the nonlinear optical compound in the core is adjusted as appropriate because of the necessity of increasing electro-optic characteristics; the amount of the nonlinear optical compound is generally 1 part by mass to 1,000 parts by mass and more preferably 10 parts by mass to 100 parts by mass relative to 100 parts by mass of the polymer compound.

[Method for Manufacturing Optical Waveguide]

The optical waveguide of the present invention is manufactured by including:

a process of forming a lower cladding using the cladding material;

a process of forming the core containing the nonlinear optical compound having a tricyano-bonded furan ring of Formula [2] or a derivative thereof on the lower cladding;

a process of forming an upper cladding using the cladding material on the core; and

a process of performing polarization orientation treatment on the nonlinear optical compound or the derivative thereof contained in the core before and/or after the process of forming the upper cladding.

More specifically, when a ridge type optical waveguide is manufactured, for example, it is manufactured through the following processes. When a slab type optical waveguide is manufactured, process (3) is performed after process (1) without passing through process (2):

(1) a process of forming the lower cladding using the cladding material;
(2) a process of forming a resist layer having photosensitivity to ultraviolet rays or an electron beam on the lower cladding, irradiating the surface of the resist layer with ultraviolet rays via a photomask or directly irradiating the surface of the resist layer with an electron beam, performing development to form a core pattern, transferring the core pattern to the lower cladding with the core pattern serving as a mask, and removing the resist layer;
(3) a process of forming the core containing the nonlinear optical compound having a tricyano-bonded furan ring of Formula [2] or the derivative thereof on the lower cladding; and
(4) a process of forming the upper cladding using the cladding material on the core.

Before and/or after process (4), the following process (5) is included:

(5) a process of performing the polarization orientation treatment on the nonlinear optical compound or the derivative thereof contained in the core.

The following a method for manufacturing the optical waveguide in detail.

<(1) Process of Forming Lower Cladding>

First, using the cladding material, a thin film (a hardened film) to be the lower cladding is formed.

Specifically, examples of the method include a method that applies the cladding material, which may be in the form of varnish (a film forming material) obtained by dissolving or dispersing the cladding material in an organic solvent as appropriate, to an appropriate substrate using a method of application such as spin coating, blade coating, dip coating, roll coating, bar coating, die coating, slit coating, ink jetting, or printing (such as letterpress, intaglio, planographic, or screen printing) and dries the applied cladding material. Among the methods of application, spin coating is preferred. Spin coating can perform application in a short time and has the advantages that even a solution with high volatility can be used and that application with high uniformity can be performed.

A method for drying the solvent is not limited to a particular method; the solvent may be evaporated in an appropriate atmosphere, that is, in air, an inert gas such as nitrogen, a vacuum, or the like using a hot plate or an oven, for example. With this process, a thin film (a hardened film) having a uniform film surface can be obtained. The drying temperature, which is not limited to a particular temperature so long as the solvent can be evaporated, is preferably 40° C. to 250° C.

The organic solvent that can be used for the film forming material is not limited to a particular solvent so long as the solvent can dissolve and/or disperse the cladding material.

Specific examples of the organic solvent include aromatic hydrocarbons such as toluene, p-xylene, o-xylene, m-xylene, ethylbenzene, and styrene; aliphatic hydrocarbons such as n-hexane and n-heptane; halogenated hydrocarbons such as chlorobenzene, ortho-dichlorobenzene, chloroform, dichloromethane, dibromomethane, and 1,2-dichloroethane; ketones such as acetone, ethyl methyl ketone, isopropyl methyl ketone, isobutyl methyl ketone, butyl methyl ketone, diacetone alcohol, diethyl ketone, cyclopentanone, and cyclohexanone; esters such as ethyl acetate, propyl acetate, isopropyl acetate, butyl acetate, isobutyl acetate, ethyl lactate, and γ-butyrolactone; amides such as N,N-dimethylformamide, N,N-dimethylacetamide, N-methyl-2-pyrrolidone, and N-cyclohexyl-2-pyrrolidone; alcohols such as methanol, ethanol, propanol, 2-propanol, allyl alcohol, butanol, isobutyl alcohol, tert-butyl alcohol, pentanol, 2-methylbutanol, 2-methyl-2-butanol, cyclohexanol, 2-methylpentanol, octanol, 2-ethylhexanol, benzyl alcohol, furfuryl alcohol, and tetrahydrofurfuryl alcohol; glycols such as ethylene glycol, propylene glycol, hexylene glycol, trimethylene glycol, diethylene glycol, 1,3-butanediol, 1,4-butanediol, and 2,3-butanediol; ethers such as diethyl ether, diisopropyl ether, tetrahydrofuran, 1,4-dioxane, ethylene glycol dimethyl ether, diethylene glycol dimethyl ether, diethylene glycol diethyl ether, and triethylene glycol dimethyl ether; glycol ethers such as ethylene glycol monomethyl ether, ethylene glycol monoethyl ether, ethylene glycol monoisopropyl ether, ethylene glycol monobutyl ether, ethylene glycol monomethyl ether acetate, ethylene glycol monoethyl ether acetate, propylene glycol monomethyl ether; propylene glycol monoethyl ether, propylene glycol monobutyl ether; propylene glycol monomethyl ether acetate, butylene glycol monomethyl ether, diethylene glycol monomethyl ether, diethylene glycol monoethyl ether, diethylene glycol monoethyl ether acetate, dipropylene glycol monomethyl ether, and dipropylene glycol monoethyl ether; 1,3-dimethyl-2-imidazolidinone; and dimethylsulfoxide. Each of these organic solvents may be used singly, or two or more of them may be used in combination.

The substrate on which the lower cladding is formed, which is not limited to a particular substrate, is preferably one having excellent planarity. Examples thereof include metallic substrates, silicon substrates, and transparent substrates, which can be selected as appropriate in accordance with the form of the optical waveguide. Preferred examples of the metallic substrates include gold, silver, copper, platinum, aluminum, and chromium. Preferred examples of the transparent substrates include substrates such as glass and plastic (poly(ethylene terephthalate) and the like).

When a lower electrode is arranged between the substrate and the lower cladding, the electrode can be a known electrode. The lower electrode may be a metal vacuum-evaporated layer or a transparent electrode layer. Preferred examples of the metal to be vacuum evaporated include gold, silver, copper, platinum, aluminum, and chromium. Preferred examples of the transparent electrode layer include indium tin oxide (ITO), fluorine doped tin oxide (FTO), and antimony doped tin oxide.

<(2) Process of Transferring Core Pattern>

Next, the resist layer having photosensitivity to ultraviolet rays or an electron beam is formed on the lower cladding, and a mask pattern of the core is formed by photolithography that irradiates the surface of the resist layer with ultraviolet rays via the photomask or directly irradiates the surface of the resist layer with an electron beam and performs development.

The resist layer, which is not limited to a particular material so long as it is a material on which microscopic patterns can be photosensitized and developed by the photolithography and in which a solvent used in the process does not elute the lower cladding, is preferably a positive type or negative type photoresist material. For a light source for pattern formation, a mercury lamp, a UV-LED, a KrF laser, an ArF laser, or the like is used.

Next, dry etching using a gas is performed with the mask pattern of the core of the resist layer serving as a mask, whereby the core pattern is transferred to the lower cladding. For this dry etching, reactive ion etching using a gas species selected as appropriate from the etching characteristics of the resist and the lower cladding, or normally CHF₃, O₂, Ar, CF₄, or the like, is preferably used.

After the dry etching, the resist layer serving as the mask is removed with a solvent.

<(3) Process of Forming Core>

Next, the core containing the nonlinear optical compound having a tricyano-bonded furan ring of Formula [2] or the derivative thereof is formed on the lower cladding on which the core pattern has been formed.

Specifically, as previously described in <Core>, examples of the method include a method that dissolves the nonlinear optical compound having a tricyano-bonded furan ring of Formula [2] and the polymer matrix material in an appropriate organic solvent with an appropriate ratio to make the form of varnish, applies the varnish to a substrate, and dries the applied varnish to form a thin film (a hardened film) and a method that dissolves a polymer compound having the derivative of the nonlinear optical compound having a tricyano-bonded furan ring of Formula [2] in its side chain in an appropriate organic solvent to make the form of varnish, applies the varnish to a substrate, and dries the applied varnish to form a thin film (a hardened film).

For a method for applying the varnish, drying conditions, and the organic solvent, those exemplified in <(1) Process of Forming Lower Cladding>can be used.

Not to elute the lower cladding during the formation of the core, the organic solvent that does not dissolve the lower cladding is selected.

<(4) Process of Forming Upper Cladding>

Using the cladding material, a thin film (a hardened film) to be the upper cladding is formed similar to <(1) Process of Forming Lower Cladding>.

<(5) Process of Performing Polarization Orientation Treatment>

Before and/or after forming the upper cladding, the polarization orientation treatment is performed by electric field poling that applies an electric field to the nonlinear optical compound contained in the core. The polarization orientation treatment is performed at a temperature near the glass transition temperature of the core or higher, orients the polarization of the nonlinear optical compound to an electric field application direction by electric field application, and holds the orientation even after the temperature is returned to room temperature, whereby electro-optic characteristics can be imparted to the core and the optical waveguide.

For the electric field application, a method that applies a DC voltage between electrodes arranged in the up-and-down direction of a stacked structure and a method using corona discharge onto the core surface are used; in view of the simplicity and the uniformity of the orientation treatment, electric field application treatment by the electrodes is preferred.

### EXAMPLES

The following describes the present invention more specifically with reference to examples. The present invention is not limited to the following examples.

Apparatuses and conditions used for the preparation of samples and analysis on properties in the examples are as follows:

### (1) Gel Permeation Chromatography (GPC)

### [Condition A]

Apparatus: HLC-8200GPC manufactured by Tosoh Corporation

Column: Shodex (registered trademark) GPC KF-804L +GPC KF-805L manufactured by Showa Denko K.K.

Column temperature: 40° C.

Solvent: THF

Detector: UV (254 nm)

Calibration line: standard polystyrene

### [Condition B]

Apparatus: HLC-8200GPC manufactured by Tosoh Corporation

Column: Shodex (registered trademark) OHpak SB-803 HQ +OHpak SB-804 HQ manufactured by Showa Denko K.K.

Column temperature: 40° C.

Solvent: DMF (with 29.6 mM of H₃PO₄, 29.6 mM of LiBr.H₂O, and 0.01% by volume of THF added)

Detector: UV (254 nm)

Calibration line: standard polystyrene

### (2) ¹H NMR Spectrum

### [PcM and PMC110-10]

Apparatus: NMR System 400NB manufactured by Agilent Technologies Japan, Ltd.)

Solvent: CDCl₃

Internal standard: tetramethylsilane (δ, 0.00 ppm)

### [PTPA-PBA-SO₃H]

Apparatus: JNM-ECA700 manufactured by JEOL Ltd.

Measurement solvent: DMSO-d₆ (deuterated dimethylsulfoxide)

Standard substance: tetramethylsilane (δ, 0.00 ppm)

### (3) Glass Transition Temperature (Tg) Measurement

Apparatus: Photo-DSC 204 Fl Phoenix (registered trademark) manufactured by Netzsch

Measurement condition: in a nitrogen atmosphere

Temperature rising rate: 30° C./minute (-50° C. to 250° C.) [PMC110-10] : 40° C./minute (25° C. to 350° C.) [PTPA-PBA]

### (4) Differential Thermobalance (TG-DTA)

Apparatus: TG-8120 manufactured by Rigaku Corporation

Temperature rising rate: 10° C./minute

Measurement temperature: 25° C. to 750° C.

### (5) Ion Chromatography (Sulfur Quantitative Analysis)

Apparatus: ICS-1500 manufactured by Dionex Corporation

Column: IonPacAG12A +IonPacAS12A manufactured by Dionex Corporation

Solvent: (2.7 mmol of NaHCO₃+0.3 mmol of Na₂CO₃)/L aqueous solution

Detector: electric conductivity

### (6) Small-Sized High-Speed Cooling Centrifuge (Centrifugal Separation)

Apparatus: SRX-201 manufactured by Tomy Seiko Co., Ltd.

### (7) UV-Visible Spectrophotometer (Absorbance Measurement)

Apparatus: SHIMADZU UV-3600 manufactured by Shimadzu Corporation

Measurement wavelength: 400 to 1,650 nm

### (8) Wet Jet Mill (Dispersion Treatment)

Apparatus: Nano Jet Pul (registered trademark) JN20 manufactured by Jokoh Co., Ltd.

### (9) Ultrasonic Washing Machine (Dispersion Treatment)

Apparatus: ASU-3M manufactured by As One Corporation

### (10) Spin Coater

Apparatus: ACT-220D manufactured by Active Co., Ltd.

### (11) Hot Plate

Apparatus: MH-3CS +MH-180CS manufactured by As One Corporation

### (12) DC Power Supply

Apparatus: Model 2410 High Voltage Source Meter manufactured by Keithley Instruments

(13) Refractive index measurement

Apparatus: multi-incident angle spectroscopic ellipsometer VASE (registered trademark) manufactured by J. A. Woollam Co., Inc.

Abbreviations represent the following meanings:

MMA: methyl methacrylate [manufactured by Tokyo Chemical Industry Co., Ltd.]
MOI: 2-isocyanatoethyl methacrylate [Karenz MOI (registered trademark) manufactured by Showa Denko K. K.]
AIBN: 2,2′-azobis(isobutyronitrile) [V-60 manufactured by Wako Pure Chemical Industries, Ltd.]
DBTDL: dibutyltin dilaurate [manufactured by Tokyo Chemical Industry Co., Ltd.]
WS-700: oxazoline-based polymer-containing aqueous solution [Epocros (registered trademark) WS-700 with a solid content of 25% by mass, a weight average molecular weight of 4×10⁴, and an oxazoline group amount of 4.5 mmol/g manufactured by Nippon Shokubai Co., Ltd.]
CNT-1: refined SWCNT [ASP-100F manufactured by Hanwha Nanotech Corporation]
CNT-2: polyethylene glycol-modified, single-walled carbon nanotube [652474-100MG manufactured by Aldrich]
SI-60L: cationic polymerization initiator [San-Aid SI-60L manufactured by Sanshin Chemical Industry Co., Ltd.]
BYK-333: polysiloxane-based surface regulator [BYK (registered trademark)-333 manufactured by BYK Japan KK]

### DMF: N,N-dimethylformamide

IPA: 2-propanol
PGME: 1-methoxy-2-propanol
THF: tetrahydrofuran

### Reference Example 1

### Manufacture of Nonlinear Optical Compound

As a nonlinear optical compound to be introduced into a side chain of a polymer, the following compound [EO-1] was used. As a nonlinear optical compound to be dispersed in a polymer matrix, the following compound [EO-2] was used. These compounds were manufactured by a technique similar to a technique disclosed in X. Zhang et al., Tetrahedron Lett., 51, p. 5823 (2010).

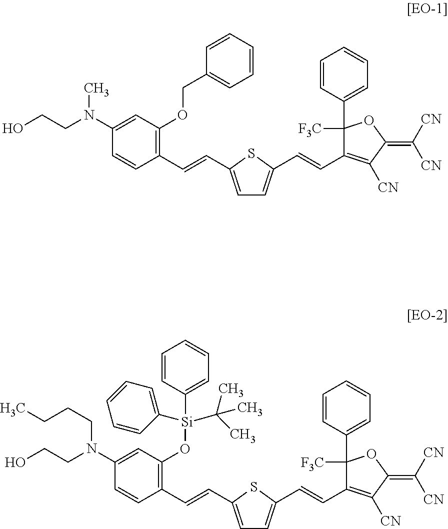 [SMILES: CN(CCO)c1ccc(/C=C/c2ccc(/C=C/C3=C(C#N)C(=C(C#N)C#N)OC3(c3ccccc3)C(F)(F)F)s2)c(OCc2ccccc2)c1 ; CCCCN(CCO)c1ccc(/C=C/c2ccc(/C=C/C3=C(C#N)C(=C(C#N)C#N)OC3(c3ccccc3)C(F)(F)F)s2)c(O[Si](c2ccccc2)(c2ccccc2)C(C)(C)C)c1]

### Manufacture Example 1-1

### Manufacture of PcM

In a nitrogen atmosphere, 10.0 g (100 mmol) of MMA, 3.87 g (25 mmol) of MOI, and 0.41 g (2.5 mmol) of AIBN were dissolved in 43 g of toluene, and the mixture was stirred at 65° C. for 3 hours. After being left to be cooled to room temperature (about 25° C.) this reaction mixture was added to 694 g of hexane to precipitate a polymer. This precipitate was filtered out and was dried under reduced pressure at room temperature (about 25° C.) to obtain 9.6 g of a white powdery target substance (PcM: refer to the following formula) (yield 69%).

FIG. 1 illustrates a ¹H NMR spectrum of the obtained target substance. The weight average molecular weight Mw of the target substance measured in terms of polystyrene by GPC (Condition A) was 46,000, and the degree of dispersion: Mw (weight average molecular weight)/Mn (number average molecular weight) was 2.1.

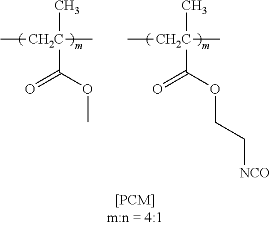

### Manufacture Example 1-2

### Manufacture of PMC110-10

In a nitrogen atmosphere, 5.7 g (8 mmol as isocyanate group) of PcM obtained in Manufacture Example 1-1, 0.63 g (0.92 mmol) of the nonlinear optical compound [EO-1] shown in Reference Example 1, and 0.38 g (0.6 mmol) of DBTDL were dissolved in 228 g of THF, and the mixture was stirred at room temperature (about 25° C.) for 88 hours.

Subsequently, 22.8 g (0.71 mol) of methanol was added thereto, and the mixture was further stirred at room temperature for 48 hours. The resultant reaction mixture was reprecipitated with 2,300 g of hexane, and the precipitate was filtered out and was dried under reduced pressure at 60° C.

The resultant solid was dissolved in 127 g of THF and was reprecipitated in 1,200 g of a heptane-ethyl acetate mixed solution (mass ratio 6:4). This precipitate was filtered out and was dried under reduced pressure at 60° C. to obtain 3.9 g of a dark green powdery target substance (PMC110-10) having a repeating unit of the following formula (yield 61%).

FIG. 2 illustrates a ¹H NMR spectrum of the obtained target substance. In PMC110-10, the content of the structure originating from the nonlinear optical compound [EO-1] was 8% by mass. The weight average molecular weight Mw of the target substance measured in terms of polystyrene by GPC (Condition B) was 88,000, the degree of dispersion (Mw/Mn) was 2.9, and the glass transition temperature Tg measured by DSC was 117.5° C.

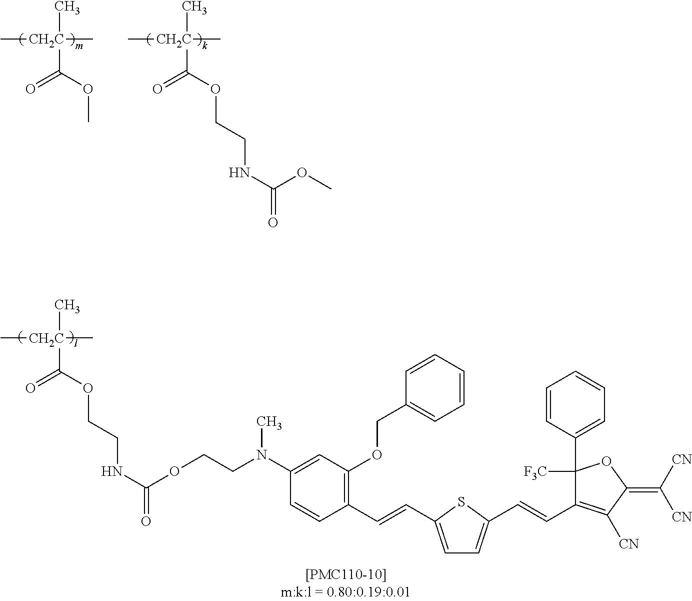

### Manufacture Example 2-1

### Manufacture of Highly Branched Polymer PTPA-PBA

In nitrogen, a 1 L four-necked flask was charged with 80.0 g (326 mmol) of triphenylamine [manufactured by Zhenjiang Haitong Chemical Industry Co., Ltd.], 118.8 g (652 mmol (2.0 equivalents relative to triphenylamine)) of 4-phenylbenzaldehyde [4-BPAL manufactured by Mitsubishi Gas Chemical Company, Inc.], 12.4 g (65 mmol (0.2 equivalent relative to triphenylamine)) of p-toluene sulfonic acid monohydrate [manufactured by Konan Chemical Manufacturing Co., Ltd.], and 160 g of 1,4-dioxane. This mixture was heated up to 85° C. and was dissolved with stirring to start polymerization. After being reacted for 6 hours, the reaction mixture was left to be cooled to 60° C. This reaction mixture was diluted with 560 g of THF, and 80 g of 28% by mass aqueous ammonia was added thereto. This reaction solution was charged into a mixed solution of 2,000 g of acetone and 400 g of methanol to be reprecipitated. The precipitated precipitate was filtered out and was dried under reduced pressure, and the resultant solid was redissolved in 640 g of THF and was charged into a mixed solution of 2,000 g of acetone and 400 g of water to be reprecipitated again. The precipitated precipitate was filtered out and was dried under reduced pressure at 130° C. for 6 hours to obtain 115.1 g of Highly Branched Polymer PTPA-PBA having a repeating unit of Formula [A] below.

The weight average molecular weight Mw of the obtained PTPA-PBA measured in terms of polystyrene by GPC (Condition A) was 17,000, and the degree of dispersion

(Mw/Mn) was 3.82. The 5% weight loss temperature measured by TG-DTA was 531° C., and the glass transition temperature Tg measured by DSC was 159° C.

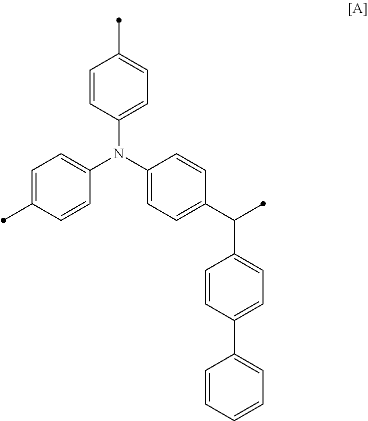

(where the black dots indicate bonding ends.)

### Manufacture Example 2-2

### Manufacture of Highly Branched Polymer PTPA-PBA-SO₃H

In nitrogen, a 500 mL four-necked flask was charged with 2.0 g of PTPA-PBA manufactured in Manufacture Example 2-1 and 50 g of sulfuric acid [manufactured by Kanto Chemical Co., Inc.]. This mixture was heated up to 40° C. and was dissolved with stirring to start sulfonation. After being reacted for 8 hours, the reaction mixture was heated up to 50° C. and was further reacted for 1 hour. This reaction mixture was charged into 250 g of pure water to be reprecipitated. The precipitate was filtered out, was added to 250 g of pure water, and was left to stand for 12 hours. The precipitate was filtered out and was dried under reduce pressure at 50° C. for 8 hours to obtain 2.7 g of Highly Branched Polymer PTPA-PBA-SO₃H (hereinafter, simply referred to as PTPA-PBA-SO₃H) as violet powder.

The sulfur atom content of PTPA-PBA-SO₃H calculated from sulfur quantitative analysis was 6.4% by mass. The sulfo group content of PTPA-PBA-SO₃H determined from this result was 1 per one repeating unit of Highly Branched Polymer PTPA-PBA (the repeating unit of Formula [A]).

### Manufacture Example 2-3

### Preparation of SWCNT Dispersion Liquid using PTPA-PBA-SO₃H

As a dispersant, 2 g of PTPA-PBA-SO₃H manufactured in Manufacture Example 2-2 was dissolved in a mixed solvent of 2.191 g of IPA and 2.806 g of pure water as a dispersion medium. To this solution, 1 g of CNT-1 as SWCNT was added. This mixture was subjected to dispersion treatment with 70 MPa and 50 passes at room temperature (about 25° C.) using a wet jet mill to obtain an SWCNT-containing dispersion liquid.

A UV-visible-near infrared absorption spectrum of the obtained SWCNT-containing dispersion liquid was measured to clearly observe the absorption of semiconducting S₁₁ band and S₂₂ band and metallic bands, which revealed that SWCNT was dispersed.

### Example 1

### Preparation of Cladding Material Composition 1

In 1.0 g (0.25 g as a polymer) of the oxazoline-based polymer-containing aqueous solution WS-700, 0.08 g of citric acid hydrate [manufactured by Junsei Chemical Co., Ltd.] was dissolved and was stirred at room temperature (about 25° C.) and was filtered by a syringe filter with a pore diameter of 0.2 μm (Cladding Material Composition 1, WS700-CA). To this solution, 0.06 g (12 μg as CNT) of the SWCNT dispersion liquid prepared in Manufacture Example 2-3 was added. The mixed solution was stirred and was treated by an ultrasonic washer for 3 minutes to prepare Cladding Material Composition A (WS700-CA-CNT1).

Similar operation was performed except that the SWCNT dispersion liquid was changed to 0.02 g (0.6 μg as CNT) of a dispersion liquid of a polyethylene glycol-modified single-walled carbon nanotube (CNT-2) to prepare Cladding Material Composition B (WS700-CA-CNT2). A CNT-2 dispersion liquid was prepared by dispersing 1.2 mg of CNT-2 in 1.0 g of water, treating the dispersion liquid by an ultrasonic washer for 30 minutes, and further diluting it 40 times.

### Example 4

### Preparation of Cladding Material Composition 2

To 10 g (2.5 g as a polymer) of the oxazoline-based polymer-containing aqueous solution WS-700, a solution in which 0.2 g of SI-60L had been mixed into 1.8 g of PGME in advance and 1.25 g of a BYK-333 aqueous solution prepared in 1% by mass in advance were added. Further, 1.82 g of water was added to this mixture, which was stirred and was filtered out by a syringe filter with a pore diameter of 0.2 μm to prepare Cladding Material Composition 2 (WS700-SI).

### Example 2

### Manufacture and Resistivity Measurement of Sample for Resistivity Measurement 1

Cladding Material Composition 1 (WS700-CA), Cladding Material Composition A (WS700-CA-CNT1), or Cladding Material Composition B (WS700-CA-CNT2) prepared in Example 1 was spin coated (1,000 rpm×60 seconds) on an ITO substrate [glass with an ITO film (sputtered product), product No.: 0008 manufactured by Geomatec Co., Ltd.], was heated on a hot plate at 110° C. for 30 minutes, and was then heated on a hot plate at 120° C. for 30 minutes to manufacture a hardened film insoluble in various kinds of organic solvents. On this hardened film, copper was deposited with a thickness of 240 nm by sputtering as an upper electrode with a diameter of 1.6 mm, which was a sample for resistivity measurement. The film thicknesses of the obtained hardened films were measured by a reflection method and are listed in Table 1.

The resistivity of the obtained sample for resistivity measurement was measured by applying a voltage using a DC power supply and measuring an electrical current value. The measurement temperatures were 24° C. and 110° C. FIG. 3 illustrates a conceptual diagram of an apparatus used for the measurement. Table 1 lists the results of the resistivity of the obtained individual hardened films. In Table 1, the electric field is a value obtained by dividing the voltage applied to each of the hardened films by the film thickness.

The refractive indexes of the hardened films manufactured using Cladding Material Composition 1, Cladding Material Composition A, and Cladding Material Composition B at a wavelength of 1.55 μm were all 1.52.

### Example 5

### Manufacture and Resistivity Measurement of Sample for Resistivity Measurement 2

Cladding Material Composition 2 (WS700-SI) prepared in Example 4 was spin coated (1,000 rpm×60 seconds) on the same ITO substrate as the one used in Example 2, was heated on a hot plate at 120° C. for 15 minutes, and was then heated on a hot plate at 150° C. for 30 minutes to manufacture a hardened film insoluble in various kinds of organic solvents. On this hardened film, gold was deposited with a thickness of 100 nm by sputtering as an upper electrode with a diameter of 1.6 mm, which was a sample for resistivity measurement. The film thickness of the obtained hardened film measured by a reflection method was 1.8 μm.

The resistivity of the obtained hardened film was measured similarly to Example 2. The measurement temperatures were 20° C., 80° C., 100° C., and 120° C. FIG. 8 illustrates the results. In FIG. 8, the electric field is a value defined in Example 2.

The refractive index of the hardened film manufactured using Cladding Material Composition 2 (WS700-SI) at a wavelength of 1.55 μm was 1.52.

As illustrated in FIG. 8, it has been revealed that there is a tendency that the resistivity decreases as the temperature increases and that the resistivity is 1×10⁸ Ωm to 1×10⁹ Ωm in the range of 100° C. to 120° C.

TABLE 1
Resistivity Measurement results

| | Film | Electric | | Resistivity (Ωm) | |
|---|---|---|---|---|---|
| | thickness | field | | 24° C. | 110° C. |
| Cladding Material | 3.9 μm | 10 | V/μm | 5.2 × 10⁸ | 6.5 × 10⁸ |
| Composition 1: | | 27 | V/μm | 2.7 × 10⁸ | 1.4 × 10⁸ |
| WS700-CA | | 101 | V/μm | 3.9 × 10⁷ | 4.2 × 10⁷ |
| Cladding Material | 2.2 μm | 1.7 | V/μm | 1.8 × 10⁶ | — |
| Composition A: | | 10 | V/μm | — | 2.4 × 10⁶ |
| WS700-CA-CNT1 | | 22 | V/μm | 2.7 × 10⁸ | 1.1 × 10⁸ |
| | | 100 | V/μm | 3.5 × 10⁷ | 6.4 × 10⁷ |
| Cladding Material | 2.5 μm | 10 | V/μm | 6.8 × 10⁸ | 6.8 × 10⁸ |
| Composition B: | | 19 | V/μm | 2.8 × 10⁸ | 2.2 × 10⁸ |
| WS700-CA-CNT2 | | 100 | V/μm | 1.0 × 10⁸ | 3.8 × 10⁸ |
| Core material | 1.3 μm | 30 | V/μm | 7.4 × 10¹⁰ | — |
| | | 103 | V/μm | 3.2 × 10⁹ | 7.6 × 10⁷ |
| Core Material B | 1.3 μm | 30 | V/μm | 8.8 × 10¹⁰ | — |
| | | 100 | V/μm | 7.1 × 10⁸ | 5.8 × 10⁷ |

As listed in Table 1, in the measurement at 24° C., in the application of a low electric field, WS700-CA and WS700-CA-CNT2 showed a resistivity of 5.2 to 6.8×10⁸ Ωm (the electric field: 10 V/μm), whereas WS700-CA-CNT1 showed 1.8×10⁶ Ωm (the electric field: 1.7 V/μm); it has been revealed that the use of the SWCNT dispersion liquid containing the dispersant prepared in Manufacture Example 2-3 significantly reduces the resistivity.

In contrast, when an electric field of 20 V/μm or stronger was applied, the effect of resistivity reduction of WS700-CA-CNT1 disappeared, and in the electric field range of 9 V/μm to 27 V/μm, all the samples showed a resistivity of 2.7 to 2.8×10⁸ Ωm.

Further, when an electric field of about 100 V/μm was applied, WS700-CA and WS700-CA-CNT1 showed a resistivity of 3.5 to 3.9×10⁷ Ωm, whereas WS700-CA-CNT2 showed 1.0×10⁸ Ωm.

The resistivity at 110° C. showed the same tendency as that of the measurement result at 24° C.

### Example 3

### Characteristics Evaluation of Optical Waveguide Modulator 1

(1) Preparation of Core Material Solution

With the nonlinear optical polymer: PMC110-10 containing 8% by mass of the nonlinear optical compound manufactured in Manufacture Example 1-2 as a polymer host, the nonlinear optical compound [EO-2] shown in Reference Example 1 was added thereto so as to be 25% by mass relative to the nonlinear optical polymer, and cyclopentanone was further added thereto to prepare a core material solution with a total concentration of the nonlinear optical polymer and the nonlinear optical compound of 15% by mass.

The resistivity of this core material was measured similarly to Example 2. Table 1 lists the results as well.

The refractive index of the core material after being hardened at a wavelength of 1.55 μm was 1.60.

(2) Manufacture of Optical Waveguide Modulator

The cladding material compositions obtained in Example 1 were used for the formation of the cladding, and a ridge type optical waveguide modulator was manufactured in accordance with the following procedure.

Metals were vacuum evaporated on a substrate (a silicon wafer) 7 in the order of chromium (50 nm), aluminum (400 nm to 500 nm), and chromium (50 nm) to manufacture a lower electrode 8 (FIG. 4: (a)).

Subsequently, using Cladding Material Composition 1 (WS700-CA), Cladding Material Composition A (WS700-CA-CNT1), or Cladding Material Composition B (WS700-CA-CNT2), a hardened film (2.6 μm) was manufactured on the same film formation and baking conditions as those of Example 2 to form a lower cladding 9 (FIG. 4: (a)).

An electron beam resist 10 Zep520A [manufactured by Zeon Corporation] was applied to the lower cladding with a thickness of 400 nm (FIG. 4: (b)), a linear waveguide pattern with a width of 4 μm and a length of 20 mm was manufactured using an electron-beam lithography apparatus, and the electron-beam resist was developed with o-xylene (FIG. 4: (c)).

With this resist pattern serving as a mask, etching was performed with a CHF₃ reactive gas using an ICP dry etching apparatus to form an inverse ridge pattern on the lower cladding 9. In this process, the etching was performed so as to give a height of a ridge (indicated by H in the drawing) of 650 nm to 700 nm (FIG. 4: (d)).

After removing the electron-beam resist (FIG. 4: (e)), the core material solution was spin coated (1,000 rpm×60 seconds) on the lower cladding 9 on which the inverse ridge pattern had been formed, was preliminarily dried on a hot plate at 95° C. for 30 minutes, and was dried at 95° C. for 48 hours in a vacuum to manufacture a core 11 (FIG. 4: (f)). The film thickness of the manufactured core 11 was 1.3 μm for all the cases.

On the core 11, using the same material as the material used for the lower cladding, a hardened film (2.6 μm) was manufactured on the same film formation and baking conditions as the manufacturing conditions of the lower cladding to form an upper cladding 12 (FIG. 4: (g)).

On the upper cladding, an upper gold electrode with a width of 0.8 mm and a length of 10 mm was deposited with a thickness of 250 nm by sputtering to form an upper electrode 13 (FIG. 4: (h)).

Finally, the both end faces of the waveguide were cut by substrate cleavage to form light incident end faces to complete an optical waveguide modulator (an optical waveguide 14).

(3) Polarization Orientation Treatment

Voltage was applied to the manufactured optical waveguide to perform the polarization orientation treatment on the nonlinear optical polymer and the nonlinear optical compound in the core 11. FIG. 5 illustrates a conceptual diagram of an apparatus used for the polarization orientation treatment. Specifically, the optical waveguide 14 was heated and held at 85° C. on a hot plate 15, and orientation treatment was performed with poling conditions with an applied voltage of 100 V and a voltage application holding time of 3 minutes via the upper electrode 13 and the lower electrode 8. Subsequently, after fixing the polarization orientation by rapid cooling, the voltage application was stopped. The optical waveguide with the orientation treatment completed was subjected to the evaluation of electro-optic characteristics described below.

For the optical waveguide, the upper and lower claddings of which were formed using Cladding Material Composition B (WS700-CA-CNT2), apart from the orientation treatment by the poling conditions, orientation treatment was performed on poling conditions with a holding temperature of 97° C. or 105° C. (the other conditions are the same as the above), and the electro-optic characteristics described below were evaluated.

(4) Electro-optic Characteristics of Optical Waveguide Modulator

Characteristic analysis on the optical waveguide modulator manufactured by (2) and (3) was performed. FIG. 6 illustrates a conceptual diagram of an apparatus used for the characteristic analysis.

As illustrated in FIG. 6, laser light with a wavelength of 1,500 nm from a laser generating apparatus 18 was made incident on an end face of the optical waveguide 14 with a light angle of 45° using a polarizer 19a using an optical fiber 20. Using a function generator 17, a triangular wave voltage was applied to the upper and lower electrodes (8 and 13). Outgoing light intensity from an end face opposite to the laser light incident end face was measured using an optical detector 21. Before being made incident on the optical detector, a −45° polarizer 19b was placed.

The outgoing light intensity obtained by the method of measurement changes in proportional to sin²(Γ/2) relative to the applied voltage (where Γ is a phase difference caused by the voltage application, and Γ is proportional to π(V/Vπ); V is the applied voltage; and Vπ is a half wavelength voltage). Given these circumstances, the phase difference Γ caused by the voltage application was analyzed using the outgoing light intensity measured by the optical detector, whereby the half wavelength voltage (Vπ) was evaluated (refer to FIG. 7).

The optical waveguide modulator having a smaller Vπ is excellent as an element with a low drive voltage. In the configuration of the optical waveguide of the present invention, to reduce Vπ, it is desirable to efficiently apply an electric field to the core layer in the poling treatment to increase the orientation of the nonlinear optical compound (an electro-optic dye).

The optical waveguide manufactured in Example 3 has a three-layer structure, in which the core layer and the cladding layer have different resistivity. Consequently, an applied voltage when a poling voltage is applied is not uniform across the core layer and the cladding layer, and a higher voltage is applied to a layer having a higher resistance, whereas a lower voltage is applied to a layer having a lower resistance.

The resistivity of the nonlinear optical polymer (PMC110-10) used for the core layer is on the order of 10⁷ Ωm at a temperature near a poling temperature (85° C. to 105° C.) in the case of 100 V/μm. It is desirable that the resistivity of the cladding layer be comparable to that of the core layer formed of the electro-optic polymer (up to about plus one order) or lower than it.

### Example 6

### Characteristics Evaluation of Optical Waveguide Modulator 2

(1) Preparation of Core Material Solution

A solution of Core Material B was prepared similarly to Example 3 except that polycarbonate [product No.: 181641 manufactured by Aldrich] was used as the polymer host in place of the nonlinear optical polymer.

The resistivity of this Core Material B was measured similarly to Example 2. Table 1 lists the results as well.

The refractive index of Core Material B after being hardened at a wavelength of 1.55 μm was 1.60.

(2) Manufacture of Optical Waveguide Modulator

A ridge type optical waveguide modulator was manufactured similarly to Example 3 except that Cladding Material Composition 2 (WS700-SI) obtained in Example 4 was used as the cladding material and the solution of Core Material B was used as the core material. The film thickness of the cladding was 2.1 μm for both the upper cladding and the lower cladding.

(3) Polarization Orientation Treatment

The orientation treatment was performed on the manufactured optical waveguide similarly to Example 3 except that the treatment temperature and the applied voltage were changed to 120° C. and 400 V, respectively.

(4) Electro-optic Characteristics of Optical Waveguide Modulator

Characteristic analysis on the optical waveguide modulator manufactured by (2) and (3) was performed similarly to Example 3.

Table 2 lists the poling conditions and the Vπ characteristic of the optical waveguide modulator of Example 3 in which the upper and lower claddings were manufactured using Cladding Material Composition 1 (WS700-CA), Cladding Material Composition A (WS700-CA-CNT1), and Cladding Material Composition B (WS700-CA-CNT2).

Table 2 lists the poling conditions and the Vπ characteristic of the optical waveguide modulator of Example 6 in which the upper and lower claddings were manufactured using Cladding Material Composition 2 (WS700-SI) as well.

Since Vπ was obtained here at the polarizing angle of the incident light of 45°, the measured value was multiplied by ⅔ from the relation of r₃₃=3r₁₃ to convert it to a TM mode Vπ characteristic.

TABLE 2
Poling conditions and half wavelength voltage (Vπ)
characteristic of optical waveguide modulators

| | | | Half | |
|---|---|---|---|---|
| | Poling conditions | | wavelength | |
| Cladding material | Temperature | Applied voltage | voltage (Vπ) | |
| Cladding Material | 85° C. | 100 V | 23 | V |
| Composition 1: | | | | |
| WS700-CA | | | | |
| Cladding Material | 120° C. | 400 V | 9.9 | V |
| Composition 2: | | | | |
| WS700-SI | | | | |
| Cladding Material | 85° C. | 100 V | 34 | V |
| Composition A: | | | | |
| WS700-CA-CNT1 | | | | |
| Cladding Material | 85° C. | 100 V | 4.8 | V |
| Composition B: | 97° C. | 100 V | 9.6 | V |
| WS700-CA-CNT2 | 105° C. | 100 V | >20 | V |

As listed in Table 2, the optical waveguide modulator in which the upper and lower claddings were formed using Cladding Material Composition B (WS700-CA-CNT2) showed the lowest half wavelength voltage characteristic.

In contrast, when Cladding Material Composition 1 (WS700-CA), the resistivity of which is comparable, was used, Vπ=23 V, which was high; when Cladding Material Composition A (WS700-CA-CNT1), which markedly changes its resistivity when a high voltage is applied, was used, Vπ=34 V.

From the foregoing, when WS700-CA-CNT2 was used for the cladding material, the optical waveguide optical modulator having a low half wavelength voltage was obtained.

### DESCRIPTION OF THE REFERENCE NUMERALS

1 Glass
2 ITO electrode
3 Sample layer (layer formed of Cladding Material Composition 1 (WS700-CA), Cladding Material Composition 2 (WS700-SI), Cladding Material Composition A (WS700-CA-CNT1), or Cladding Material Composition B (WS700-CA-CNT2))
4 Upper electrode
5 DC power supply
6 Ammeter
7 Substrate (silicon wafer)
8 Lower electrode
9 Lower cladding
10 Electron beam resist
11 Core
12 Upper cladding
13 Upper electrode
14 Optical waveguide
15 Hot plate
16 DC power supply
17 Function generator
18 Laser generating apparatus
19 (19a, 19b) Polarizer
20 Optical fiber
21 Optical detector
22 Oscilloscope

1. A cladding material of an optical waveguide comprising a polymer compound containing an oxazoline structure in a side chain and an acid generator or a polycarboxylic acid.

2. The cladding material of an optical waveguide according to claim 1, comprising the polymer compound containing an oxazoline structure in a side chain and the acid generator.

3. The cladding material of an optical waveguide according to claim 1, comprising the polymer compound containing an oxazoline structure in a side chain and the polycarboxylic acid.

4. The cladding material of an optical waveguide according to claim 1, comprising the polymer compound containing an oxazoline structure in a side chain, a carbon nanotube, and the acid generator or the polycarboxylic acid.

5. The cladding material of an optical waveguide according to claim 3, comprising the polymer compound containing an oxazoline structure in a side chain, a carbon nanotube, and the polycarboxylic acid.

6. The cladding material of an optical waveguide according to claim 1, wherein the polymer compound is obtained by radically polymerizing at least two kinds of monomers of an oxazoline monomer having a polymerizable carbon-carbon double bond-containing group at the 2-position of an oxazoline ring and a hydrophilic functional group-containing (meth)acrylic monomer.

7. An optical waveguide comprising a core and a cladding that surrounds an entire outer periphery of the core and has a refractive index lower than that of the core, the cladding being formed of the cladding material as claimed in claim 1.

8. The optical waveguide according to claim 7, wherein
the core contains an organic nonlinear optical compound having a tricyano-bonded furan ring of Formula [2] or a derivative thereof: 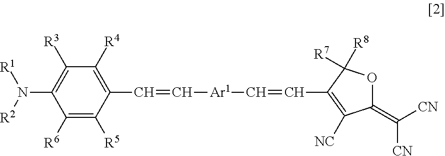
(where R¹ and R² are each independently a hydrogen atom, a C₁₋₁₀ alkyl group optionally having a substituent, or a C₆₋₁₀ aryl group optionally having a substituent; R³ to R⁶ are each independently a hydrogen atom, a C₁₋₁₀ alkyl group, a hydroxy group, a C₁₋₁₀ alkoxy group, a C₂₋₁₁ alkylcarbonyloxy group, a C₄₋₁₀ aryloxy group, a C₅₋₁₁ arylcarbonyloxy group, a silyloxy group having a C₁₋₆ alkyl group and/or phenyl group, or a halogen atom; R⁷ and R⁸ are each independently a hydrogen atom, a C₁₋₅ alkyl group, a C₁₋₅ haloalkyl group, or a C₆₋₁₀ aryl group: and Ar¹ is a divalent aromatic group of Formula [3] below or Formula [4] below): 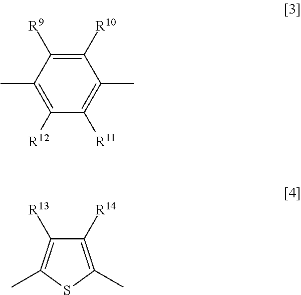
(where R⁹ to R¹⁴ are each independently a hydrogen atom, a C₁₋₁₀ alkyl group optionally having a substituent, or a C₆₋₁₀ aryl group optionally having a substituent).

9. A method for manufacturing the optical waveguide as claimed in claim 8 including a core and a cladding that surrounds an entire outer periphery of the core and has a refractive index lower than that of the core, the method comprising:
forming a lower cladding using the cladding material of an optical waveguide comprising a polymer compound containing an oxazoline structure in a side chain and an acid generator or a polycarboxylic acid;
forming a core containing the nonlinear optical compound having a tricyano-bonded furan ring of Formula [2] or the derivative thereof as claimed in claim 8 on the lower cladding;
forming an upper cladding using the cladding material on the core; and
performing polarization orientation treatment on the nonlinear optical compound or the derivative thereof contained in the core before and/or after the forming the upper cladding.

10. A method for manufacturing the ridge type optical waveguide as claimed in claim 8 including a core and a cladding that surrounds an entire outer periphery of the core and has a refractive index lower than that of the core, the method comprising:
forming a lower cladding using the cladding material of an optical waveguide comprising a polymer compound containing an oxazoime structure in a side chain and an acid generator or a polycarboxylic acid;
forming a resist layer having photosensitivity to ultraviolet rays or an electron beam on the lower cladding, irradiating a surface of the resist layer with ultraviolet rays via a photomask or directly irradiating a surface of the resist layer with an electron beam, performing development to form a mask pattern of the core, transferring a core pattern to the lower cladding with the mask pattern serving as a mask, and removing the resist layer;
forming a core containing the nonlinear optical compound having a tricyano-bonded furan ring of Formula [2] or the derivative thereof as claimed in claim 8 on the lower cladding;
forming an upper cladding using the cladding material on the core; and
performing polarization orientation treatment on the nonlinear optical compound or the derivative thereof contained in the core before and/or after the forming the upper cladding.

11. The method for manufacturing according to claim 9, wherein the polarization orientation treatment is electric field application treatment by electrodes.
